(19) United States
(12) Patent Application Publication
(10) Pub. No.: US 2007/0059386 A1
(43) Pub. Date: Mar. 15, 2007

(54) Modular system for novel Chinese herbal formulas

(76) Inventor: Tsun-Nin Lee, San Francisco, CA (US)

Correspondence Address: Tsun-Nin Lee, 1790 26th Avenue, San Francisco, CA, 94122, US

(21) Appl. No.: 11/516,327

(22) Filed: Sep. 7, 2006

Related U.S. Application Data
(60) Provisional application No. 60/716,205, filed on Sep. 12, 2005.

(51) Int. Cl. A61K 36/18

(52) U.S. Cl. 424/725

(57) ABSTRACT

A modular system for novel Chinese herb formulas that allows for a practitioner skilled the basic principles of Traditional Chinese Medicine to employ a limited number of herbal formulations in various combinations to create safe custom treatments for individual patients for a wide variety of medical conditions.

### CROSS-REFERENCE TO RELATED APPLICATIONS

This invention is entitled to the benefit of Provisional Patent Applications Ser. No. 60/716,205 filed 2005, Sep. 12.

### BACKGROUND—FIELD OF INVENTION

This invention relates to herbal compositions for the treatment of medical conditions; specifically, to a modular system of herbal formulations allowing for custom treatments for a wide variety of medical indications.

### BACKGROUND—DESCRIPTION OF PRIOR ART

In contrast to Western countries, in eastern Asia, particularly in China, herbal therapies have been common throughout several millennia. For many diseases, Chinese herbal therapies may increase the effectiveness of modern drug treatments, reduce their side effects, or replace them completely.

The basis for the diagnosis and treatment of disease under the system of Traditional Chinese Medicine (TCM) relies heavily upon the concepts of Yin and Yang as well as the Five Elements—Earth, Metal, Water, Wood, and Fire and the importance of their balance. Although there is no Western equivalent to these terms, and such monikers may seem to relate more to mysticism than science, these terms actually provide a very logical and reliable construct to classify etiologies of disease and guide correct treatment approaches. The strength of this system is evidenced by thousands of years of empirical yet undeniable success in the management of a panoply of medical conditions.

TCM herbal therapies are typically given in combinations of individual herbs and have been shown to be superior to single agent therapies. Unlike in Western medicine, the same disease is often treated differently for different people due to variations in constitution and specific symptoms. Therefore, TCM is highly customized for particular individuals.

However, the proficient use of herbal remedies typically requires years of dedicated analysis and practice given the vast myriad of single agents available. For example, there are over 400 common herbal agents and many more substances used less frequently. Furthermore, even if a health care practitioner is skilled in the use of most agents in the pharmacological armamentarium, keeping the requisite inventory of herbs in a typical medical practice is generally not practical, given storage and cataloging requirements.

Consequently, the system proposed in this application was created as a simpler, yet extremely effective modular approach to allow a medical practitioner the ability to treat a wide variety of medical conditions and restore balance and harmony to the body without the necessity to create complicated custom formulas de novo.

The concept of modular formulation has been presented before; one of the earliest noted efforts was by a physician named Sun Simiao around the 7^{th} century A.D. He stipulated that therapeutic formulas could be interpreted as the combination of modular components that work synergistically. While this concept helps with understanding the mechanism of formulas, it does not notably reduce the complexity of the TCM herbal treatment, as there were still countless modular components.

Practitioners have also, over the centuries, applied single or multiple herb modifications to popular formulas in order to modify treatments for individual patients, but this strategy does not employ a true modular system with broad applicability.

Until to the development of this present invention, there has not been a satisfactory system to simplify and substitute the traditional custom application of individual herbs.

### SUMMARY

In accordance with the present invention, a modular system for novel Chinese herb formulas allows for a limited number of herbal formulations to be combined uniquely for individual patients in order to treat a wide variety of medical conditions.

### OBJECTS AND ADVANTAGES

Accordingly, several objects and advantages of the present invention are the following:
(a) to provide a modular system of herbal formulations which may be uniquely combined to treat a variety of medical conditions in different individuals
(b) to provide a modular system with a limited number of herbal formulations to reduce storage and inventory requirements for a comprehensive herbal pharmacy
(c) to provide a modular system of herbal formulations that will allow healthcare practitioners with a basic understanding of TCM to deliver effective treatments to patients without requiring intimate knowledge of individual constituent components of the formulas.
(d) to provide a modular system of herbal formulations that is safe to use

### DRAWING FIGURES

None

### DESCRIPTION

Presented in this application are herbal compositions that provide treatment for a wide range of medical conditions, promote health, and prevent disease. In particular, these formulas are designed to be uniquely combined in a modular manner to optimize treatment for specific conditions. This modular system thus allows for more a flexible yet comprehensive approach compared with the customary manner of practicing traditional Chinese Herbal Medicine.

Herbal compositions as defined in this application shall include any natural products including botanicals, animal products, or minerals.

This modular system of herbal formulae consists of three separate groups: G-series, S-series, and M-series. The G-series represents a group of general tonification formulae; the S-series herbal formulae have specific actions on the various systems of the body according to TCM; and the M-series formulae have the capacity to modify the other formulae; Some of the characteristics of the herbal formulae within this modular system are outlined as follows:
(a) Their functions are similar to historically popular formulae in some cases, but are more balanced in nature.
(b) Some of the formulae may be similar to certain historically popular formulae, but are more potent, in order to accomplish their balancing actions.
(c) The total number of herbal formulae in the modular system is rather limited, allowing the practitioners to store these herbs in their offices quite conveniently.
(d) The formulae may be combined in various ways to yield new formulae. This can be easily accomplished by mixing the capsules from various formulae as the capsules have already been pre-proportioned for this purpose.
   Formulae of the G Series:
   Special Note of the G Series:

The members of this series regulate as well as tonify. Cosmosol, Neptonin, and Noren are three formulae that focus their attention on the kidney system. Among the three, Cosmosol has the most global effect. Neptonin has greater action on kidney Yin, while Noren has greater action on kidney Yang. Both Pancremax and Girotate work on the Central qi or the spleen-stomach system, although Pancremax has a stronger tonification effect than Girotate, while Girotate has a stronger regulating effect than Pancremax. Both Diamonex and Octogen work on the gallbladder-liver system. The former is more tonifying, while the latter is more regulating, although they both have tonifying and regulating qualities. Three major systems, namely, the kidney, liver and spleen are covered by the seven members of the G series.

1. Cosmosol.

A. Composition of Formula:

| | | |
|---|---|---|
| Yam Rhizome | Dioscorea Batatis Rhizoma | Shan Yao |
| Jujube fruit | Zizyphi Fructus | Da Zao |
| Cassia twig | Cinnamomi Ramulus | Gui Zhi |
| Rehmannia cured root tuber | Rehmanniae Radix Proeparata | Gan Di |
| | | huang |
| Hyacinth bean | Dolichoris Semen | (bai)Bian |
| | | Dou |
| Dong quai root | Angelicae Sinensis Radix | Dang Gui |
| Barley sprouts | Hordei Germinatus Fractus | Mai Ya |
| Ophiopogon root tuber | Ophiopogonis Tuber | Mai Men |
| | | Dong |
| Asian ginseng root | Panax | Ren Shen |
| Bai-zhu atractylodes | Atractylodisiore | Bai Zhu |
| | Macrocephalae Rhizoma | |
| Poria sclerotium | Poriae Cocos | Fu Ling |
| Peony root without bark | Paeoniae Radix | Shao Yao |
| Sichuan lovage rhizome | Cnidii Rhizoma (Ligustici | Chuan |
| | Rhizoma) | Xiong |
| Bupleurum root | Bupleuri Radix | Chai Hu |
| Apricot seed | Armeniacae Semen | Xing Ren |
| Platycodon root | Platycodi Radix | Jie Geng |
| Licorice root | Glycyrrhizae Radix | Gan Cao |
| Donkey-hide gelatin | Asini Gelatinum | E Joao |
| Siler root | Ledebouriellae Radix | Fang Feng |
| Schisandra fruit | Schizandrae Fructus | Wu Wei Zi |
| Ginger rhizome (dried) | Zingiberis Siccatum Rhizoma | Gan Jiang |

B. Functions and Actions:

The primary function of this formula is general tonification. Unlike some other tonification formulae, such as the Perfect Ten, which focuses on mainly the spleen system and the liver system, Cosmosol works on all systems. The formula Perfect Ten is enriching but tends to be somewhat stagnating, while Cosmosol tonifies as well as regulates each system. Cosmosol therefore has a greater action range than the various other formulae with tonification properties. For instance, Ren Shen, Bian Dou, Bai zhu and Shan Yao tonify the spleen while Mai Ya and Fu Ling mobilizes and decongests the stomach and spleen. By doing so, the tonification process does not lead to congestion of the spleen.

In summary, this formula possesses the following actions:
1. Tonify kidney Yin: Gan Di Huang, E Jiao
2. Tonify and regulate the liver: Dang Gui Shao Yao, Chuan Xiong, Chaihu and Fang Feng
3. Tonify and regulate the spleen Shan Yao, Bian Dou, Ren Shen, Bai Zhu, Fu Ling and Mai Ya.
4. Tonify and regulate the lung system: Mai Men Dong, Xing Ren and Jie Geng.
5. Tonify the center: Gancao, Gan Jiang and dazao.
6. Mobilize qi and the enterior: Gui Zhi and Fang Feng
   C. Similarity to Old Formulae:

A formula found in the Important Strategies of the Golden Chest contains most of the ingredients in Cosmosol. However, some key ingredients have been replaced for Cosmosol.

D. Inappropriate Use of Formula:

Although Cosmosol is both tonifying and regulating in nature, it is generally a bit more tonifying. Therefore, in individuals with congestive states, this formula may not be appropriate.

2. Neptonin.

A. Composition of Formula:

| | | |
|---|---|---|
| Rehmannia cured root tuber | Rehmanniae Radix | Shu Di |
| | Proeparata | Huang |
| Rehmannia unprocessed | Rehmanniae Radix | Sheng Di |
| root tuber | | Huang |
| Yam rhizome | Dioscorea Batatis Rhizoma | Shan Yao |
| Asiatic dogwood fruit | Corni Fructus | Shan Zhu |
| without seed | | Yu |
| Tree peony root bark | Moutan Radicis Cortex | Mu Dan Pi |
| Poria sclerotium | Poriae Cocos | Fu Ling |
| Asian water plantain rhizome | Alismatis Rhizoma | Ze Xie |
| Lycium fruit | Lycii Fructus | Gou Qi Zi |
| Cuscuta | Cuscutae Semen | Tu Si Zi |
| Barley dried germinated | Hordei Germinatus Fructus | Mai Ya |
| ripe fruit | | |
| Donkey-hide gelatin | Asini Gelatinum | E Joao |

B. Functions and Actions:

The primary function of Neptonin is to tonify kidney Yin, although it has the capabilities of tonifying Yang as well. Both Gou Qi Zi and Tu Si Zi tonify kidney and liver Yin, whereas E Jiao strengthens Yin and blood. Therefore, this formula is good for many Yin deficiency states. Both Shu Di Huang and Sheng Di Huang tonify the kidney system. Fu Ling and Ze Xie direct the Qi to the urogenital system. Shan Yao and Shan Zhu Yu together tonify the spleen system, which being the earth element, can suppress the water element. Finally Mu Dan Pi will eliminate the excessive heat due to Yin deficiency. By reducing the degree of Yin deficiency, this formula can counteract the symptoms of floating Yang, or pseudo-Yang. As many medical conditions are associated with Yin deficiency states. This formula may be quite helpful in many such conditions.

C. Similarity to Old Formulae:

A formula similar to Neptonin known as Liu Wei Di Huang Wan is also found in the Important Strategies of the Golden Chest. However, Neptonin has an even stronger Yin tonifying function than Liu Wei Di Huang Wan. The tonifying nature of the Liu Wei Di Huang Wan is sometimes stagnating. The addition of Mai Ya to Neptonin by and large eliminates this problem.

D. Inappropriate Use of a Formula:

This formula will not be useful in individuals with a predominantly Yin constitution, with symptoms of Yang deficiency.

E. Why the New Formula:

This formula is more specifically Yin tonifying, yet at the same time less stagnating than Liu Wei Di Huang Wan.

3. Noren:

A. Composition of Formula:

| | | |
|---|---|---|
| Rehmannia cured root tuber | Rehmanniae Radix | Gan Di |
| | Proeparata | huang |
| Rebmannia unprocessed | Rehmanniae Radix | Sheng De |
| root tuber | | (Huang) |
| Yam rhizome | Dioscorea Batatis Rhizoma | Shan Yao |
| Asiatic dogwood fruit without | Corni Fructus | Shan Zhu |
| seed | | Yu |
| Tree peony root bark | Moutan Radicis Cortex | Mu Dan Pi |
| Poria sclerotium plantain | Poriae Cocos | Fu Ling |
| rhizome | | |
| Psoralea fruit | Psoraleae Semen | Bu Gu Zhi |
| Morinda root | Morindae Radiz | Ba Ji Tian |
| Cassia twig | Cinnamomi Ramlus | Gui Zhi |
| Barley sprouts | Hordei Germinatus Fractus | Mai Ya |
| Hawthorn fruit | Crataegi Fructus | Shan Zha |

B. Functions and Actions:

Noren tonifies both kidney Yin and kidney Yang, although it is most effective in activating Yang in someone with a clinical picture of Yang deficiency. Several ingredients in this formula overlap those of Neptonin, so it has Yin tonifying properties as well. The three ingredients, Bu Gu Zhi, Ba Ji Tian and Gui Zhi are primarily Yang tonifying.

C. Similarity to Old Formula:

This formula is similar to the formula known as the Golden Chest Kidney Qi Pill, except that Fu Zi has been replaced with Bu Gu Zhi and Ba Ji Tian.

D. Why this New Formula:

Some individuals, despite their Yang deficiency, sometimes cannot tolerate the potent Yang tonifying effect of Fu Zi, and may develop symptoms of excessive heat. It also tends to stagnate the energy, causing stuffiness in the stomach, or a feeling of indigestion. The addition of Mai Ya and Shan zha solves this problem. The tendency of Fu Zi to overshoot the yang tonification process has been moderated by replacing Fu Zi with Bu Gu Zhi and Ba Ji Tian.

E. Inappropriate Use of Formula:

In individuals with marked symptoms of Yin deficiency, care must be exercised to avoid too much tonification of the Yang. Since this formula is tonifying in nature, even though the formula has been modified, its use should be limited in conditions characterized by stagnation or congestion.

4. Diamonex:

A. Composition of Formula:

| | | |
|---|---|---|
| Dong quai root | Angelicae Sinansis Radix | Dang Gui |
| Rehmannia cured root tuber | Rehmanniae Radix Proeparata | Gan Di |
| | | Huang |
| Peony root without bark | Paeoniae Radix | Shao Yao |
| Bupleurum root | Bupleuri Radix | Chai Hu |
| Sichuan lovage rhizome | VCnidii Rhizoma (Ligustici | Chuan |
| | Rhizoma) | Xiong |
| Sichuan teasel root | Dipsaci Radix | Xu Duan |
| Drynaria rhizome | Drynariae Rhizoma | Gu Sui Bu |

B. Functions and Actions:

The primary function of this formula is to tonify as well as to regulate the liver-gallbladder system. Dang Gui is an herb used to tonify the liver and blood. Shao Yao tonifies and regulates liver Yin. Sheng Di Huang tonifies the kidney. Chai Hu specializes in regulating the liver-gallbladder system while Chuan Xiong is a mobilizer of the energy in the liver-gallbladder system. Xu Duan and Gu Sui Bu are also regulators and tonifiers for the liver and gallbladder. The whole formula therefore focuses on the liver gallbladder system. It tonifies but at the same time disperses or mobilizes the energy within this system. It is generally good for someone who is deficient in both liver Yin and Yang, yet it decreases the tendency to stagnate. The versatility of this formula is a direct result of the special composition of this formula.

C. Similarity to Old Formulae:

A popular herbal formula known as the Four Substances Decoction has been used extensively in traditional Chinese medicine.

D. Why this New Formula:

Dang Gui is particularly enriching in nature and sometimes causes indigestion. Composed of Dang Gui and Shu Di Huang, the four substances decoction may prove to be too warm and too “rich” for some individuals. The addition of Chai Hu cools off this formula to some degree and spread the qi along the gallbladder-liver system (Shao Yang). Since the liver-gallbladder system also controls the tendons and sinews, it is generally involved in trauma. Both Xu Duan and Gu Sui Bu has been used in traditional Chinese medicine to promote healing of bones and tendons. The addition of these agents into the formula helps to potentiate the normalizing effects of this particular formula on the gallbladder-liver system.

E. Inappropriate Use of Formula:

Individuals with liver-gallbladder system congestion are less likely to be benefited by this formula. It is also warming in nature despite the addition of Chai Hu. Therefore this formula is not applicable in conditions where liver heat dominates.

5. Pancremax:

A. Composition of Formula:

| | | |
|---|---|---|
| Codonopsis root | Codonopsis Pilosulae Radix | Dang Shen |
| Bai-zhu atractylodes | Atractylodis Macrocephalae | Bai Zhu |
| | Rhizoma | |
| Astragalus root | Astragali Radix | Huang Qi |
| Longan fleshy seed cover | Longanae Arillus | Long Yan |
| | | Rou |
| Poria pararadicis sclerotium | Poria Spirit (Tuckahoe Spirit) | Fu Shen |
| Jujube seed | Zizyphi Spinosi Semen | Suan Zao |
| | | Ren |
| Costus root | Aucklandia Lappa Radix | Mu Xiang |
| Ginger rhizome (fresh) | Zingiberis Rhizoma | Sheng Jiang |
| Jujube fruit | Zizyphi Fructus; | Da Zao |
| Licorice root | Glycyrrhizae Radix | Gan Cao |
| Dong quai root | Angelicae Sinansis Rodix | Dang Gui |
| Polygala root | Polygalae Radix | Yuan Zhi |
| Polygonatum rhizome | Polygonati Rhizoma | Huang Jing |
| Euryale seed | Euryalis Semen | Qian Shi |

B. Functions and Actions:

This formula specializes in tonifying the spleen system. Among the spleen tonifying agents in this formula are: Dang Shen, Bai zhu, dazao, Huang Jing, Qian Shi and Gancao. Among the agents that tonify and activate the heart system are: Fu Shen and Long Yan Rou. The element of wood is also activated to produce more heart fire by including the following: Dang Gui, Yuan Zhi, Sheng Jiang, Suan Zao Ren. Huang Qi and Mu Xiang is added to mobilize the qi. By and large this formula simultaneously tonifies the wood, fire, and earth elements. Consequently, it tends to suppress the water element, which often dominates in cold and wet conditions. It also possesses a generally warming effect. Because of its earth element strengthening action, it can definitely tonify the stomach and spleen. Furthermore, it can dispel dampness in the gastrointestinal tract.

C. Similarity to Old Formula:

This formula resembles the TCM formula as Gui Pi Tang which has been used traditionally to treat many chronic conditions, including GI disturbances, menstrual dysfunctions, and other problems associated with spleen qi deficiency.

D. Why this New Formula:

Although this formula is similar to Gui Pi Tang, its spleen tonifying properties are even stronger with the addition of Huang Jing and Qian Shi.

E. Inappropriate Use of the Formula:

Any condition where the stomach qi and heart qi is congested and where signs of excessive heat are present.

6. Girotate:

A. Composition of Formula:

| | | |
|---|---|---|
| Asian ginseng root | Panax Ginseng | Ren Shen |
| Bai-zhu atractylodes | Atractylodis Macrocephalae | Bai Zhu |
| | Rhizoma | |
| Poria sclerotium | Poriae Cocos | Fu Ling |
| Yam rhizome | Dioscorea Batatis Rhizoma | Shan Yao |
| Hyacinth bean | Dolichoris Semen | (Bai)Bian |
| | | Dou |
| Licorice root | Glycyrrhizae Radix | Gan Cao |
| Platycodon root | Platycodi Radix | Jie Geng |
| Ginger rhizome (fresh) | Zingiberis Rhizoma | Sheng |
| | | Jiang |
| Jujube fruit | Zizyphi Fructus | Da Zao |
| Tangerine dried rind of | Citri Pericarpiump | Chen Pi |
| mature fruit | | |
| Bitter orange nearly mature fruit | Citri Aurantii Fructus | Zhi Qiao |

B. Functions and Actions:

This formula works primarily on the GI tract, as it both tonifies and regulates the stomach and spleen system. According to TCM, Ren Shen (ginseng), Bai zhu, Gancao, Shan Yao, Bian Dou and Da Zao will all tonify the spleen and stomach. Fu Ling percolates the wetness away from the spleen, while Chenpi mobilizes qi. Sheng Jiang stirs and moves qi. Zhi Qiao disperses congestion in the stomach, while Jie Geng ventilates the energy out of the lung system. While this formula tonifies the stomach and spleen system, it does not allow the qi to remain stagnant.

C. Similarity to Old Formula:

The composition of Girotate is similar to that of a number of traditional Chinese medicine formulae, particularly the Four Gentlemen Decoction and the Six Gentlemen Decoction, which are well-known formulae to tonify to spleen and stomach.

D. Why this New Formula:

This formula regulates as well as tonifies in contrast to the Four Gentlemen Decoction, which is more tonifying than regulating. This property imparts to this formula greater flexibilities in dealing with imbalances within the gastrointestinal system. Since the element of earth produces metal, the activation of the lung system by Jie Geng allows excessive energy to be ventilated from the stomach-spleen system, reducing stagnation. To further deal with the potential congestion of energy in the stomach, Zhi Qiao is employed. Both Chen Pi and Fu Ling are used to transform phlegm. Since the earth generates metal, this formula may also be useful when the lung qi is weak. As stagnation, weakness of spleen qi and phlegm is present in many of the chronic conditions, this formula may be quite versatile in handling all such imbalances.

E. Inappropriate Use of the Formula:

Due to the balanced nature of this formula, they are relatively few instances in which the use of this formula is inappropriate. However, caution still has to be exercised when there are problems of stagnation.

7. Octagen:

A. Composition of Formula:

| | | |
|---|---|---|
| Bupleurum root | Bupleuri Radix | Chai Hu |
| Trichosanthes root | Trichosanthis Radix | Gua Lou Gen |
| Cassia twig | Cinnamomi Ramulus | Gui Zhi |
| Chinese skullcap root | Scutellariae Radix | Huang Qin |
| Ginger (dried) | Zingiberis Siccatum Rhizoma | Gan Jiang |
| Oyster shell | Ostreae Concha | Mu Li |
| Licorice root | Glycyrrhizae Radix | Gan Cao |
| Astragalus root | Astragali Radix | Huang Qi |
| Codonopsis root | Codonopsis Pilosulae Radix | Dang Shen |

B. Functions and Actions:

The primary function of octagen is to regulate the liver-gallbladder system. One of the key ingredients is Chai Hu, which guide the therapeutic effects of other agents to the liver-gallbladder system. Gan Jiang or dry ginger is a hot agent, while Huang Qin is cold. This combination of hot and cold regulates the temperature of the channel. Mu Li and Gua Lou Gen promote the liver Yin. To equalize the energy between the stomach and the gallbladder, Huang Qi and Dang Shen are employed to engender the stomach qi. This formula is good for individuals whose constitution may be so weak that they cannot tolerate other kinds of tonifications. Since the gallbladder-liver system primarily deals with stress, it is usually weakened in chronic illnesses. Stimulating and regulating the system can potentially modify the stress response.

C. Similarity to Old Formula:

This formula is similar to Chai Hu Gui Zhi Gan Jiang Tang.

D. Why this New Formula:

Many individuals who are weak in qi should welcome the strengthening of their stomach qi by the addition of Huang Qi and Dang Shen.

E. Inappropriate Use of Formula:

Conditions primarily unrelated to the gallbladder-liver system.

IV. Formulae of the S Series:

Special Note About the S Series:

As a general rule, the formulae in this series guide the herbal agents to the target systems or organs where imbalances exist. These formulae simultaneously employ tonification agents as well as dispersing agents in order to regulate the system in question.

1. Lumbagon:

A. Composition of Formula:

| | | |
|---|---|---|
| Astragalus root | Astragali Radix | Huang Qi |
| Codonopsis root | Codonopsis Pilosulae Radix | Dang Shen |
| Yam rhizome | Dioscorea Batatis Rhizoma | Shan Yao |
| Lycium fruit | Lycii Fructus | Gou Qi Zi |
| Eucommia stem bark | Eucommiae Cortex | Du Zhong |
| Achyranthes root | Achyranthis Radix | Niu Xi |
| Tienchi ginseng root | Notoginseng Radix | San Qi |
| Job's Tears seed | Coicis Semen | Yi Yi Ren |
| Euryale seed | Euryalis Semen | Qian Shi |
| Morinda root | Morindae Radix | Ba Ji Tian |
| Jujube fruit | Zizyphi Fructus | Da Zao |

B. Functions and Actions:

This formula tonifies the kidney system, particularly when it relates to the lower half of the body. It also tonifies the spleen system, which is the earth element, to suppress excessive water. The spleen tonification agents include Da Zao, Dang Shen, Shan Yao and Qian Shi. In order to guide the herbal effects to the lower half of the body, Niu Xi is used. San Qi along with Niu Xi has the ability to disperse blood stasis. Gou Qi Zi and Du Zhong strengthen the tendons and muscles by tonifying the liver system. Huang Qi, on the other hand, elevates the in general. Yi Yi Ren is used to dispel the wetness. Ba Ji Tian, Du Zhong and Huang Qi are all Yang agonists. The whole formula tends to be warm, drying, Yang tonifying, and qi replenishing for the lower half of the body. Individuals suffering from low backache and sciatica often have the constitution of excessive cold and wetness due to spleen qi deficiency and kidney qi deficiency.

C. Similarity to Old Formula:

In traditional Chinese medicine, many formulae have been used to treat back pain and sciatica. Some of the ingredients in this formula are found in such formulae. But the combination of the ingredients in this formula is unique.

D. Why this New Formula:

This formula approaches the imbalance found in lowback pain in a multifaceted way by tackling the various systems at the same time: counteracting the coldness, dampness, kidney deficiency, excessive phlegm, weakness of the tendons by focusing on the lower half of the body.

E. Inappropriate Use of this Formula:

Most low back pain is due to kidney qi deficiency and in the majority of instances, symptoms of kidney Yang deficiency dominates. However, it should be kept in mind that some conditions of back pain and leg pain are due to excessive heat or pseudo-Yang due to deficiency of Yin. This formula, therefore, should not be used in these situations without modifications of the formula, for example, by combining with the modifyer Arctin, which has a cooling effect.

2. Nasoclear:

A. Composition of Formula:

| | | |
|---|---|---|
| Fragrant angelica root | Angelicae Dahuricae Radix | Bai Zhi |
| Mint aboveground parts | Menthae Herba | Bo He |
| Codonopsis root | Codonopsis Pilosulae Radix | Dang Shen |
| Bai-zhu atractylodes | Atractylodis Macrocephalae | Bai Zhu |
| | Rhizoma | |
| Magnolia flower bud | Magnoliae Flos | Xin Yi Hwa |
| Xanthium fruit | Xanthii Fructus | Cang Er Zi |

B. Functions and Actions:

This formula focuses on action in the facial area, particularly the nose and throat. This is accomplished by the herbs Cang Er Zi and Xin Yi Hwa. This combination is assisted by Bai Zhi, which is a specialist of the Yang Ming system that encompasses the large intestine and stomach meridians that travel to the head, controlling the functions of the oral cavity, nasal cavity and the face. Since the nasopharynx may be considered as an extension of the lungs, which in turn control the exterior, Bo He is capable of dispelling excessive energy in the exterior, accompanied by cooling effects. It works well with the other agents. Both Bai zhu and Dang Shen, on the other hand, provide tonifying effects to mobilize the lung system.

C. Similarity to Old Formula:

In the armamentarium of TCM, some formulae used Cang Er Zi and Xin Yi Hwa to treat nasal conditions of various kinds.

D. Why this New Formula:

As mentioned above, most of the TCM formulae dealing with nasal congestion, rhinorrhea, and allergy symptoms mainly utilize herbal agents to disperse the exterior. However, the employment of stomach tonification agents such as Bai zhu and Dang Shen will actually give this formula great impetus to disperse with a great deal more efficiency. The temperature of this formula is neither hot nor cold. Many individuals with chronic rhinitis or multiple allergies suffer from spleen qi deficiency, so formulae that emphasize on dispersing the exterior are in fact qi depleting and are less likely to help them in the long run. This formula contains herbal agents to strengthen the stomach system, so it is more likely to achieve the ultimate goal of balancing the internal environment.

E. Inappropriate Use of the Formula:

Generally, this formula is quite neutral.

3. Serena:

A. Composition of Formula:

| | | |
|---|---|---|
| Bupleurum root | Bupleuri Radix | Chai Hu |
| Os Draconis | Draconis Os | Long Gu |
| Oyster shell | Ostreae Concha | Mu Li |
| Large-leaf gentian root | Gentianae Macrophyllae | Qin Jiao |
| | Radix | |
| Lycium root bark | Lycii Radaicis Cortex | Di Gu Pi |
| Rehmannia unprocessed root | Rehmanniae Radix | Sheng De |
| tuber | | Huang |
| Rehmannia cured root tuber | Rehmanniae Radix Proeparata | Gan Di |
| | | huang |

B. Functions and Actions:

One of the outstanding effects of this formula is to anchor the floating Yang resulting from Yin deficiency. A disturbance of the liver-gallbladder system often leads to anxiety and even agitation. That's why one of the major ingredients of this formula is Chai Hu. Qin Jiao also enters into the Yin portion of the gallbladder-liver system and can assist in guiding the herbal effects of other components of the formula to that system. Both Long Gu and Mu Li have the special properties of the holding down the floating Yang, being Yin substances themselves. Both Shu Di Huang and Sheng Di Huang are known for their actions to potentiate the kidney Yin. Additionally, Sheng Di Huang is much more cooling in nature. Historically, Di Gu Pi has been used to treat conditions of bones steaming states, typified by symptoms of Yin deficiency. Therefore, this formula is extremely helpful in controlling the unbridled Yang.

C. Similarity to Old Formula:

Many of the agents in this formula have been found in other TCM formulae to achieve the effects of calming the Yang, but not necessarily in the combination proposed in this formula.

D. Why this New Formula:

The combination of agents as outlined in this formula achieves synergistic effects not available with the individual agents separately. To compensate for the cooling effects of the majority of the agents, Shu Di Huang is added to provide warmth and protect the body from losing energy by replenishing the kidney qi.

E. Inappropriate Use of the Formula:

Despite the presence of Shu Di Huang, this formula is cold in character, so it should be avoided in individuals suffering from Yang deficiency states. The cold nature of this formula may be modified by adding other formulae with a warming nature.

4. Cephalicon:

A. Composition of Formula:

| | | |
|---|---|---|
| Dong quai root | Angelicae Sinansis Rodix | Dang Gui |
| Bai-zhu atractylodes | Aractylodis Macrocephalae Rhizoma | Bai Zhu |
| Chinese lovage root & rhizome | Ligustici Sinensis Rhizoma et Radix | Gao Ben |
| Round-leaf chaste tree fruit | Viticis Fructus | Man Jing Zi |
| Sichuan lovage rhizome | Cnidii Rhizoma (Ligustici Rhizoma) | Chuan Xiong |
| Bitter orange nearly matured fruit | Citri Aurantii Fructus | Zhi Qiao |
| Skullcap root | Scutellariae Radix | Huang Qin |
| Ginger rhizome (dried) | Zingiberis Siccatum Rhizoma | Gan Jiang |
| Tangerine dried rind of mature fruit | Citri Pericarpium | Chen Pi |
| Licorice root | Glycyrrhizae Radix | Gan Cao |
| Fragrant angelica root | Angelicae Dahuricae Radix | Bai Zhi |

B. Functions and Actions.

The purpose of this formula is to direct its effects to the head in order to normalize its functions. The two main ingredients deployed for this objective are Gao Ben and Man Jing Zi. Bai Zhi activates and regulates the Yang Ming meridians that affect the frontal region of the head. Gancao and Bai Zhu tonify the stomach, while Zhi Qiao gets rid of excessive energy in the system. Dang Gui and Chuan Xiong, on the other hand, tonify and mobilize the qi within the liver and gallbladder meridians, which also travel to the head area. Gan Jiang warms up, while Huang Qin cools down the formula. Chen Pi serves the function of mobilizing qi in general. The net effect of this formula is to reestablish homeostasis in the head.

C. Similarity to Old Formula:

Some of the herbal ingredients in the formula have been used in the past in China to treat problems in the head.

D. Why this Formula:

This formula contains both hot and cold agents so as to normalize temperature in the region, as headaches may be related to either excessive heat or cold. Many headaches are associated with imbalances of energy in the head due either to the overbearing stomach or liver system in the stomach-liver dichotomy. This formula tends to normalize both the stomach and liver systems. To prevent trapping of energy within the systems, this formula employs agents to disperse as well as to mobilize qi.

E. Inappropriate Use of the Formula:

Although this formula is quite balanced different individuals may respond to the formula differently. Therefore, clinical judgment needs to be exercised.

5. Royala:

A. Composition of Formula:

| | | |
|---|---|---|
| Wheat seed | Tritici Fructus | Fu Xiao Mai |
| Astragalus root | Astragali Radix | Huang Qi |
| Oyster shell | Ostreae Concha | Mu Li |
| Licorice root | Glycyrrhizae Radix | Gan Cao |
| Jujube fruit | Zizyphi Fructus | Da Zao |

B. Functions and Actions:

The main action of this formula is to prevent the floating Yang from activating the exterior. When there is a significant deficiency in qi, the pseudo Yang, uncontrolled by Yin, which is also deficient, will rise up to the exterior, causing symptoms such as flushing and sweating. In order to strengthen the interior so as to anchor the qi this formula employs Dazao and Gancao. Mu Li, an agent of known for its ability to anchor the floating Yang is also used. Huang Qi, which can engender qi and also strengthen the exterior, is an essential ingredient of this formula. Fu Xiao Mai has been historically used to treat hot flashes in China and is also part of the formula.

C. Similarity to Old Formula:

Gan Mai Da Zao Tang is a formula used traditionally to treat hot flashes.

D. Why this New Formula:

Although Gan Mai Da Zao Tang has been a useful formula, it does not specifically address the over all qi deficiency state found in many individuals suffering from hot flashes. Specifically, Huang Qi addresses the issue of global qi deficiency. Mu Li, which also has an outstanding ability to anchor the floating Yang is also added to the formula.

E. Inappropriate Use of the Formula:

This formula is quite mild in nature, but the strong central qi tonification may not be suitable for someone who has stomach qi stagnation.

6. Relaxol:

A. Composition of Fformula:

| | | |
|---|---|---|
| Bupleurum root | Bupleuri Radix | Chai Hu |
| Dong quai root | Angelicae Sinansis Rodix | Dang Gui |
| Peony root without | Paeoniae Radix | Shao Yao |
| bark | | |
| Bai-zhu atractylodes | Atractylodis Macrocephalae | Bai Zhu |
| | Rhizoma | |
| Poria sclerotium | Poriae Cocos | Fu Ling |
| Licorice root (baked) | Glycyrhizae Radix Praeparata | Zhi Gan Cao |
| Tangerine dried rind of | Citri Immaturi Pericarpium | Qing Pi |
| green fruit | | |
| Tangerine dried rind of | Citri Pericarpium | Chen Pi |
| mature fruit | | |
| Mint | Menthae Herba | Bo He |

B. Functions and Actions:

This formula is intended to accomplish the great balancing act between the stomach and the liver system. Chaihu, Dang Gui and Shao Yao work on the liver system, while Bai zhu, Fu Ling and Zhi Gan Cao work on the stomach system. These agents simultaneously tonify and regulate. The end result is that neither the stomach nor the liver system will dominate the other, reaching a state of harmony. Chen Pi mobilizes qi in the stomach system, while Qing Pi mobilizes qi in the liver system. These qi moving agents prevent the tonifiers from causing stagnation within the systems.

C. Similarity to Old Formula:

This formula is a similar to the Rambling Powder that has been used in traditional Chinese medicine to treat irritability, emotional lability, anxiety, etc.

D. Why this New Formula:

Both Bai zhu and Dang Gui in the Rambling Powder are quite tonifying in nature and may cause stagnation in some instances. Qing Pi would prevent the stagnation due to Dang Gui and Chenpi will prevent Stagnation caused by Bai zhu. The regulatory effects of these two simple agents actually improve the efficacy and reduce the side-effects of the formula.

E. Inappropriate Use of the Formula:

Most of the ingredients in this formula tend to be warm, and therefore this formula should be avoided in individuals with excessive Yang or pseudo-Yang conditions.

7. Eumensa:

A. Composition of Formula:

| | | |
|---|---|---|
| Dong quai root | Angelicae Sinansis Rodix | Dang Gui |
| Peony root without bark | Paeoniae Radix | Shao Yao |
| Rehmannia cured root tuber | Rehmanniae Radix Proeparata | Shu Di |
| | | Huang |
| Astragalus root | Astragali Radix | Huang Qi |
| Licorice root | Glycyrrhizae Radix | Gan Cao |
| Melia fruit | Meliae Toosendan Fructus | Chuan Lian |
| | | Zi |
| Cattail pollen | Typhae Pollen | Pu Huang |
| Fennel fruit | Foeniculi Fructus | Xiao Hui |
| | | Xiang |
| Corydalis yanhusuo tuber | Corydalis Yanhusuo Rhizoma | Yan Hu Suo |
| Sichuan lovage rhizome | Cnidii Rhizoma | Chuan Xiong |
| | (Ligustici Rhizoma) | |

B. Functions and Actions:

This formula regulates functions in the pelvic region and the genital area mainly controlled by the liver meridian. The general tonification of the liver meridian is accomplished by the four substances: Shu Di Huang, Chuan Xiong, Shao Yao and Dang Gui. Huang Qi adds more qi to the formula. Xiao Hui Xiang is an herbal agent that mobilizes qi in the Lower Jiao (lower focus of the body). Both Pu Huang and Yan Hu Suo are decongestants of the liver meridian with Pu Huang's main action in the pelvic region. Chuan Lian Zi, on the other hand, is an activator of a segment of the liver meridian in the lower part of the body. Finally, Gancao is used to harmonize the entire formula.

C. Similarity to Old Formula:

This formula contains the Four Substances Decoction, which has been used traditionally in Chinese medicine to treat a variety of women's conditions.

D. Why this New Formula:

This formula contains several agents that direct the qi to the pelvic region governed by the liver meridian. The addition of the four substances decoction therefore tonify the liver meridian in the pelvic area. Simultaneously, other agents that decongest the liver meridian are also employed. As a whole this formula mobilizes qi and blood in the lower abdominal area with concurrent tonification. It therefore has the ability to normalize functions in this region according to the principles of TCM.

E. Inappropriate Use of this Formula:

As a precaution, do not use this formula during pregnancy.

V. Formulae of the M Series:

Special Note on the M Series:

Special attention has to be paid to the M-series herbal formulae, because the members of this series are unlike the G-Series and S-series of herbal formulae, which are quite balanced in nature. The functions of the M-series are by necessity unidirectional. In other words, there is no hedging for these formulae. They are not meant to be taken alone. If the diagnosis is not made correctly, it will push the physiological balance in the wrong direction. For instance, if someone suffering from cold syndrome is mistakenly thought to have hot syndrome and given Arctin as a result, his cold syndrome will be made worse.

The second point to remember about the M-Series is that they should be used in small quantities only. In most instances, only one capsule of the M. series members should be used in each dosing, generally in combination with about five capsules of the G-Series or S-Series, which contain in their formulae some tonification components, so as to prevent the depletion of qi.

All five members of the M-series have the potential of consuming qi. The most potent member of the M-series is Detonin, for its main action is to disperse blood stasis. As compared to the other members of this group, it is also most qi consuming. To measure qi consumption, let us use the qi consumption index. The value of 1 is assigned to the qi consumption index of Detonin. Less potent than Detonin are Destagnate and Acceler and the qi consumption index is assigned a value of between 0.5 and 0.75. The least potent in terms of qi consumption among the five members of the M. series are Arctin and Heleon. These are given the value of 0.25 to 0.5. When more than one modifier is employed in the same dose, the qi consumption index should be added up to estimate the combined potency of the two modifiers. For example, when Arctin and Destagnate are used together, the qi consumption index is approximately from a low of 0.75 (0.25+0.5) to a high of 1.25 (or 0.5+0.75). The ratio between the number of capsules of a G-Series or S-Series member and the combined qi consumption index of the modifiers should be maintained at 3:1 or higher to prevent qi depletion. In most cases, a 4:1 ratio is appropriate. For instance, when I Diamonex Acceler, and Detonin a combined to create a new formula, one capsule each of Detonin and Acceler together will yield a consumption index of 1+0.50 or 1.5. Therefore, five capsules of Diamonex should be combined with these two agents to maintain a ratio of greater than 3:1. Furthermore, the M. series members should not be combined to create a new formula without the participation of the tonification formulae.

1. Arctin:

A. Composition of Formula:

| | | |
|---|---|---|
| Asparagus root tuber | Asparagi Tuber | Tian Men Dong |
| Ophiopogon root tuber | Ophiopohonis Tuber | Mai Men Dong |
| Dendrobium stem | Dendrobii Caulis | Shi Hu |
| Glehnia root | Glehniae Radix | Bei Sha Shen |
| | cum Rhizoma | |
| Coptis rhizome | Coptidis Rhizoma | Huang Lian |
| Scullcap root | Scutellariae Radix | Huang Qin |
| Cortex Phellodendron amurense | Phellodendri Cortex | Huang Bo |

B. Functions and Actions:

The primary purpose of this formula is to cool. Huang Lian is effective in cooling the upper focus of the body. Huang Qin cools off the middle jiao, while Huang Bo lowers the temperature of the Lower Jiao. Bei Sha Shen is capable of replenishing the yin fluid. Shi Hu is a general yin tonification agent and is therefore cooling in nature. Both Tian Men Dong and Mai Men Dong are herbal agents that tonify the metal element or the lung system. Since metal generates water, these two agents also preserve the Yin fluid, which counteracts heat.

C. Similarity to Old Formula:

Historically, many herbal formulae in Chinese medicine employed a variety of cooling of herbs. The ingredients found in Arctin have been used in the large number of formulae, although the composition of Arctin is unique.

D. Why this New Formula:

Many herbal agents of cold nature exert their effects by neutralizing Yang. When that happens, the kidney Yin will be tapped to replenish the neutralized Yang. So in the long run, the body's vital energy, both Yin and Yang, will be depleted. This formula utilizes both the powerful agents to cool off the excessive heat and the strong Yin intoxication agents to preserve the Yin, to avoid depleting the body's qi. This modifier formula can be quite useful in reducing the temperature of other formulae.

E. Inappropriate Use of Formula:

This formula is not to be used in someone suffering from a Yin condition or an advanced qi deficiency state.

2. Heleon:

A. Composition of Formula:

| | | |
|---|---|---|
| Psoralea fruit | Psoraleae Semen | Bu Gu Zhi |
| Desert broomrape stem | Cistanchis Herba | Rou Cong Rong |
| Epimedium aboveground parts | Epimedii Herba | Yin Yang Huo |
| Sichuan aconite cured lateral | Aconiti Carmichaeli | (Zhi) Fu Zi |
| root | Praeparata Radix | |
| Ginger rhizome (dried) | Zingiberis Siccatum | Gan Jiang |
| | Rhizoma | |
| Eucommia stem bark | Eucommiae Cortex | Du Zhong |
| Clove flower bud | Caryophylli Flos | Ding Xiang |

B. Functions and Actions:

The primary purpose of this formula is to warm the body. Fuzi, Bu Gu Zhi and Yin Yang Huo tonify the kidney Yang. Rou Cong Rong increases total body heat, particularly heat in the stomach-spleen system. Gan Jiang is both hot and drying. Du Zhong is warm and able to tonify both the kidney and liver. Ding Xiang is an aromatic herb with a strong warming and qi mobilizing effect that particularly activates the heart meridian. As a whole this formula synergistically generates a great deal of heat.

C. Similarity to Old Formula:

Each individual herb in this formula has been known to possess warming effects and has been employed in many TCM formulae. However, they have not been combined according to the Heleon formula.

D. Why this New Formula:

All the herbal agents in this particular formula work hand-in-hand to produce heat and activate the kidney Yang. Not only is it Yang energy producing, but it is also qi mobilizing. It activates the Yang component in stomach, kidney, liver and heart. While each ingredient is effective alone, together they work better.

E. Inappropriate Use of Formula:

This formula is to be avoided in anyone with either excessive heat or pseudo-heat symptoms or signs. In individuals with severe qi deficiency, this formula must be temporized with Yin engendering components, to avoid upsetting the delicate balance in such individuals. Keep in mind that excessive use of warming agents can also deplete the body's energy storage.

3. Acceler:

A. Composition of Formula:

| | | |
|---|---|---|
| Tangerine dried rind | Citri Immaturi Pericarpium | Qing Pi |
| of green fruit | | |
| Costus root | Aucklandia Lapa Radix | Mu Xiang |
| Cyperus rhizome | Cyperi Rhizoma | Xiang Fu (Zi) |
| Lindera root | Linderae Radix | Wu Yao |
| Cassia twig | Cinnamomi Ramlus | Gui Zhi |

B. Functions and Actions:

The primary purpose of this formula is to mobilize qi. Gui Zhi essentially mobilizes qi in all meridians, but more particularly in the lung and bladder meridians that govern the exterior of the body. Qing Pi and Xiang Fu mobilize qi in mainly the liver-gallbladder system, while Mu Xiang mobilizes qi in the stomach-spleen system. Wu Yao, on the other hand, is a qi mobilizer in the Lower Jiao. Therefore, as a whole, this formula has a global qi mobilizing effect.

C. Similarity to Old Formula:

The Six Congestion Pill developed by Zhu Dan Xi centuries ago contains a number of phlegm dispersing and qi mobilizing agents to treat various states of congestion and stagnation. Acceler utilizes a combination of different agents that are mainly aromatic nature.

D. Why the New Formula:

Out of the five elements, this formula focuses on three of the most important elements: water, wood and earth or kidney, liver and spleen. The combined actions of these mobilizers have a greater global effect on the body. When added to another formula, it can exert an anti-stagnating effect.

E. Inappropriate Use of Formula:

Due to its mobilizing effects, this formula is by nature qi consuming. As a rule, it should not be used alone, but rather in conjunction with formulae with qi tonification properties. Since qi mobilizers are generally hot in nature, they activate the Yang and may produce excessive heat. This formula should be avoided in individuals with excessive Yang or deficiency of Yin.

4. Destagnate:

A. Composition of Formula:

| | | |
|---|---|---|
| Hawthorn fruit | Crataegi Fructus | Shan Zha |
| Barley dried germinated | Hordei Germinatus Fructus | Mai Ya |
| ripe fruit | | |
| Pinellia rhizome | Pinelliae Tuber | Ban Xia |
| Tangerine dried rind of | Citri Pericarpium | Chen Pi |
| mature fruit | | |
| Mustard seed | Fructus Perillae Frutescenyis | Bai Jie Zi |
| Kelp | Ulvae Thallus | Kun Bu |
| Areca husk | Arecae Pericarpium | Da Fu Pi |

B. Functions and Actions:

The main function of this formula is to disperse phlegm as well as food accumulation. A premier agent to transform phlegm is Ban Sha. The function of Chen Pi is to mobilize qi in the gastrointestinal tract. Bai Jie Zi, which is warming in nature, is used to disperse wet phlegm. To get rid of excessive water (dilute phlegm), Da Fu Pi is used. For food accumulation syndrome, the combination of Shan Zha and Mai Ya is highly effective. Kun Bu is kelp. It is an agent specialized in dispersing hot phlegm.

C. Similarity to Old Formula:

Different agents contained in this present formula have been used in traditional Chinese medicine to treat various conditions of excessive phlegm and food accumulation syndromes. Er Chen Tang is composed of Chen Pi and Ban Sha, a popular formula for transforming phlegm.

D. Why this New Formula:

Food accumulation syndrome is in fact a form of phlegm syndrome due to overstimulation of the GI tract by excessive food intake. This kind of phlegm tends to be more tenacious in nature. The addition of Shan Zha and Mai Ya are employed for this specific purpose. This formula is composed of a number of herbal agents that deal with different kinds of phlegm, hot and cold, thick and thin.

E. Inappropriate Use of Formula: Never use this formula alone in someone who is weak in stomach qi or generally qi deficient.

5. Detonin:

A. Composition of Formula:

| | | |
|---|---|---|
| Peach seed | Persicae Semen | Tao Ren |
| Safflower flower | Carthami Flos | Hong Hua |
| Corydalis yanhusuo tuber | Corydalis Yanhusuo Rhizoma | Yan Hu Suo |
| Tienchi ginseng root | Notoginseng Radix | San Qi |
| Frankincense oleo-gum- | Olibanum Gummi | Ru Xiang |
| resin | | |
| Myrrh oleo-gum-resin | Myrrha | Mo Yao |

B. Functions and Actions:

The primary purpose of this formula is to unclog blood stasis. Both Tao Ren and Hong Hua are effective to achieve this goal. San Qi is not only an an anti-blood stasis agent but also capable of normalizing the functions of blood—it can prevent clotting as well as bleeding. Yan Hu Suo tends to clean out stagnated blood. Finally, Mo Yao and Ru Xiang, being aromatics, possess the properties of mobilizing blood, or moving qi within the blood. The above agents work together to open up channels blocked by static blood.

C. Similarity to Old Formula:

Tao Ren and Hong Hua often team up together in traditional Chinese medicine formulae to treat blood stasis, whereas Ru Xiang and Mo Yao are often combined to treat blocked up energy, which causes furuncles, carbuncles and nonhealing wounds.

D. Why this New Formula:

Putting Tao Ren and HongHua, Ru Xiang and Mo Yao together creates a powerful team to unclog blocked channels. San Qi, on the other hand, provides a balancing and harmonizing effect. Yan Hu Suo further consolidates the power of this combination.

E. Inappropriate Use of this Formula:

Due to this formula's energy busting nature, it should be avoided in pregnancy as well as individuals who have severe qi deficiency. It should always be employed with qi tonification formulae to achieve its full potential. This formula should not be used alone, and when used, in small amount only.

Operation:

The following sections will illustrate the practical application of the modular system using the treatment of specific conditions under a variety of different circumstances as examples. However, the scope of utility for this system is not limited to the uses outlined hereafter.

Associated with each medical condition is an internal environment of the body that has been disturbed or pushed off balance in one way or another. The prime objective of traditional Chinese medicine is to reestablish the balance internally regardless of the causes of such imbalances. A number of commonly encountered medical problems are presented in this manual simply to illustrate the approach in problem solving, and as such, these are not the only conditions that can be benefited by the application of the modular system.

The versatility of the modular system is contingent upon a clear understanding of the patient's constitution, as well as the nature of the internal imbalance. The analytical approach is simplified by using the eight categories for diagnosis, which include Yin, Yang, hot, cold, exterior, interior, congestion, and deficiency, as well as the interactions of the Five Elements.

While employing the modular system, keep in mind the commonly espoused principles in traditional Chinese medicine: treat multiple conditions with a single formula and treat a single condition with multiple formulae. Consequently, for a specific condition, more than one primary formula may be employed, or several formulae can be recombined to yield a new formula for this purpose. Likewise, the same formula may be used over and over in what appear to be totally different conditions from the diagnostic point of view of Western modern medicine. This is because the different medical conditions share the same pathological pathways from the viewpoint of TCM. Using this theoretical as well as practical framework, a number of symptoms as well as common disorders will be analyzed.

Fatigue

This is one of the ten most frequent complaints presented at the doctor's office. A more expanded list of complaints associated with fatigue may include tiredness, weakness, poor motivation, malaise, decreased exercise tolerance, and others. Such complaints are usually nonspecific and may be associated with a number of severe and not so severe conditions. Fatigue may be acute or chronic. Acute fatigue is generally self-limiting and treatment is directed towards the underlying cause. Chronic fatigue, however, posts a much greater challenge to physicians. The symptoms of fatigue are usually accompanied by many chronic disease states, some of which are rather serious or even life-threatening. On the other hand, many individuals suffering from chronic fatigue do not have clear-cut medical diagnoses and their conditions have been lumped under the diagnosis of chronic fatigue syndrome. Fortunately, fatigue that is associated with medical illnesses and fatigue due to chronic fatigue syndrome share a great deal of common features that can lend themselves to intervention by the TCM approach.

Based on TCM, fatigue may be caused by Qi deficiency or Qi congestion, regardless of the modern medical diagnoses. Fatigue caused by Qi deficiency is similar to a car running out of gas, as there is not enough energy to keep the body running efficiently. Fatigue caused by Qi congestion, on the other hand, is a similar to a car with its fuel line clogged or its fuel injection system malfunctioning as a result of a blockage. While either Qi deficiency or Qi congestion may lead to chronic fatigue, Qi deficiency and congestion are by no means mutually exclusive, and often coexist in the same patient. In general, however, deficiency plays a dominant role in an overwhelming majority of individuals suffering from chronic fatigue of various etiologies. As corrective actions may be implemented by various tonification formulae of Chinese medicine, such problems become much more manageable using the TCM approach than the rather limited armamentarium available in modern medicine.

Algorithm for Managing Chronic Fatigue:

1. Initiate Cosmosol to take advantage of its global tonification effect. If a patient responds well to this regimen, stay on it for a while.
2. Alternatively, either Noren or Neptonin may be used, depending on whether the patient is suffering from predominantly Yin or Yang deficiency. For instance, if the patient has hypersomnulence and sensitivity to cold environment, a pale looking wet tongue, and a deep and weak pulse along with other symptoms and signs of Yang deficiency, then Noren should be used. If the patient is highly irritable and anxious with insomnia, warmth in the palms and soles, a red tongue without coating, and other signs and symptoms of a Yin deficiency state, then Neptonin should be used. If the symptoms and signs are not so clear-cut for either Yin deficiency or Yang deficiency, or if the symptoms of Yin deficiency and Yang deficiency coexist, a balanced approach may therefore be implemented by using both the formulae Neptonin and Noren. For example, two capsules of Noren and two capsules of Neptonin two times a day, or three capsules each of Noren and Neptonin two times a day may be prescribed. If there are more Yang deficiency symptoms than Yin deficiency symptoms, the ratio of Noren and Neptonin may be altered to reflect that, for instance, three capsules of Noren with two capsules of Neptonin each time.
3. Chonic fatigue syndrome sometimes coexists with fibromyalgia with the dominant symptoms of myalgia and gastrointestinal disturbances; so in this case, the right choice may be Pancremax or Girotate to improve the Stomach's functions (Earth) in order to dry up the excessive amount of Water in the body.
4. If the patient is old and debilitated, and does not handle the above formulae well, then a course of Octagen may be indicated.
5. If the above approaches are insufficient to achieve the intended goals, attention must be focused on specific organ systems that may be the culprit for the physiological imbalances. The Gallbladder-Liver system is often perturbed, giving rise to a whole host of symptoms that are indistinguishable from those of chronic fatigue syndrome or other significant chronic illnesses. The question to ask is whether the Gallbladder-Liver system is deficient or congestive. Try to ascertain the circumstances surrounding the symptom of fatigue. If the chronic fatigue is concurrent with the onset of menopause, or subsequent to a major physical drama as in an automobile accident, there is an increased likelihood that the trouble is the result of a deficient Gallbladder-Liver system. It goes without saying, however, that one must ascertain whether other diagnostic criteria such as those obtained by pulse diagnosis and tongue diagnosis are consistent with the clinical findings. If the Gallbladder-Liver system is in fact deficient, Diamonex would be indicated. If blood stasis is also present, the addition of Detonin to Diamonex may be necessary. Adding one capsule of Detonin to four or five capsules of Diamonex in each dose may be appropriate.
6. If there are multiple symptoms that relate to either the Gallbladder-Liver system or the Spleen system (as it is clinically difficult to distinguish the influences from these two major systems), then a trial course of Relaxol, which tonifies and regulates both the Liver and the Spleen system, may be reasonable. Alternatively, a half-and-half mixture of Diamonex (Gallbladder-Liver system) and Pancremax or Girotate (Spleen system) may be utilized. If the resultant new formula is too stagnating, one capsule of Destagnate may be added to each dose.
7. Keep in mind that several formulae in the S Series may also be enlisted to combat chronic fatigue depending on the clinical circumstances. For example, if chronic nasal allergy is a prominent feature in a particular patient who concomitantly suffers from chronic fatigue, the use of Nasoclear may be quite appropriate.
   General Principles in the Management of Chronic Fatigue:
1. The symptomatology in chronic fatigue may be quite complex and involve multiple organ systems. Therefore, it is unreasonable to expect instant and complete response with any kind of therapeutic intervention. Herbal formulae are not like modern drugs; adequate time is required to exert their influences on the body's physiology. Having said that, it is not at all unusual to see positive effects within a couple of weeks. If the symptoms continue to abate without concomitant ill effects, than the effective formulae should be continued for weeks or even months.
2. The resolution of symptoms may not be complete, so a partial response is still quite desirable. Stay with the effective regimen to keep the responsive symptoms at bay for a while to prevent their reoccurrence. When this objective is accomplished, then tackle the remainder of the symptoms.
3. Other than the symptoms of fatigue, patients may have other comorbidities such as insomnia and gastrointestinal complaints which are often regarded as equally troublesome. These complaints may actually guide the physician in his search for the root cause of the disease state. For instance, if GI symptoms figure prominently in the patient's symptom complex, the Spleen-Stomach system may be the source of the chronic drainage of kidney Qi. If the patient has poor dietary habits, consuming large amounts of fatty food for extended period of time, and has a thick grimy nodular coating on his tongue, Girotate plus Destagnate may be a good bet.
4. If a formula is effective in controlling certain symptoms, but is accompanied by some undesirable effects, then the formula should be changed or modified. For example, a patient who has been diagnosed to have scleroderma suffers from chronic fatigue and regains a great deal of his energy from taking Noren, but develops some degree of anxiety and more frequent awakenings at night, may benefit from the addition of Serena to his therapeutic regimen. If there is no jitteriness during the day, then add Serena to only the evening dose of Noren.
   Headaches

The following clinical entities defined by modern medical nomenclature will be analyzed based on TCM:

A. Migraine headaches.

B. Tension type headaches

C. Cluster headaches.

D. Headaches of muscular origin.

E. Miscellaneous painful conditions of the head and face.

A. Migraine Headaches

Migraine headaches are either hot or cold in nature. Based on the five elements analysis, hot headaches are the result of overactivity of Wood and Fire or Fire and Earth. When there is so much Fire and Earth, Wood is relatively weak. When there is too much Wood and Fire, Wood is relatively excessive. Therefore, hot headaches can be subclassified into the Wood excessive and Wood deficient types. When the migraine headaches are cold in nature, the patient may have a constitution that is excessive in Earth and Metal, Metal and Water, or Water and Wood. Once again, the cold migraines can be subclassified into Wood excessive and Wood deficient types. Too much Earth and Metal and too much Metal and Water tend to be accompanied by a deficiency of Wood, where as excessive amounts of Water and Wood will strengthen Wood. The classification may be summarized in the following table:

Hot migraines, Wood excess

Hot migraines, Wood deficiency

Cold migraines, Wood excess

Cold migraines, Wood deficiency.

Algorithm for Managing Migraine Headaches:

1. Cephalicon may be tried first, because it has already incorporated in its formula the basic strategies of managing migraines through the regulation of commonly involved meridians.
2. Add Arctin to Cephalicon, if the headaches are hot. Add Heleon if the headaches are cold. For starters, keep the Cephalicon to modifier ratio to about four.
3. If the headaches tend to cluster around menstruation, then kidney and liver Qi deficiency probably exist and Neptonin or Noren may be employed depending upon whether the patient has Yin deficiency or Yang deficiency. Alternatively, Diamonex may be used to tonify and regulate the Gallbladder-Liver system to stave off the occurrence of headaches.
4. If severe menstrual cramps are associated with headaches and there are other signs and symptoms of blood stasis, particularly with reference to the pelvic region, a trial of Eumensa may be indicated. In fact, Eumensa alone without Cephalicon may be sufficient.
5. If the GI symptoms associated with migraines are due to excessive Earth (deficient Wood), the use of an Earth strengthening formula such as Pancremax may increase the symptoms. But if the GI symptoms are associated with increased Water element or Wood element, Pancremax may prove to be extremely helpful. If in doubt, a few doses of Girotate may be used as a test, because Girotate is more balanced in nature.
6. Since success or failure in the management of migraines is largely dependent on accurately assessing the body's constitution, it is necessary to use all diagnostic tools available in TCM, including a careful history, which should encompass TCM review of systems along with pulse and tongue diagnoses, etc.
7. Unilateral symptoms and signs usually indicate a pathological involvement of the Gallbladder-Liver system. Hemicrania is such a condition. Within the Merideon formulary, the formulae that primarily act on the Gallbladder-Liver system include Diamonex, Octagen, Serena, Relaxol, and Eumensa. The “nemesis” of the Gallbladder-Liver is of course the Spleen-Stomach, which is the organ targeted by several other formulae, such as Girotate, Pancremax, and Nasoclear. All these formulae can be combined in various ways to achieve the ultimate balance between the Gallbladder-Liver and Spleen-Stomach systems. They can also be enlisted to balance the abnormal physiological states underlying migraine headaches, subject to the clinical findings.
   B. Tension Type Headaches

Tension type headaches can be categorized into hot and cold as well. Tension type headaches were previously classified as muscular contraction headaches, and not infrequently activated by stress. Since the Gallbladder-Liver system is an anti-stress apparatus for the body, it is often involved.

Algorithm for Managing Tension Type Headaches:

1. Cephalicon should be tried first. This formula can be used during the acute phase of headache. It may also be used continuously to adjust the internal environment, making it “less hospitable” for the headaches to occur.
2. If response to Cephalicon is incomplete or inadequate, the primary meridian that is at fault should be determined. In the great majority of instances, only the Stomach meridian and the Gallbladder meridian will be involved. The other meridians that may be at fault include the Liver meridian which goes behind the eyes or the Bladder meridian and Governor Vessel that travel primarily to the back of the head. The site of pain may be quite helpful to select the right formula.
If frontal, consider the use of Girotate or Pancremax.
If temporal, consider the use of Diamonex and Octagen.
3. Look for other clues to improve the Formulae. For example, heaviness in the head associated with tiredness and clouded thinking is often caused by excessive phlegm generated by the Stomach, so Destagnate may be added to the formula. On the other hand, if associated with extreme irritability, insomnia, and other symptoms rising out of excessive Fire, Arctin may be called into action. If there is bloating in the stomach along with indigestion and thick wet oily coating (white, not yellow) caused by the food accumulation syndrome, Acceler may then be added along with Destagnate. A simple formula is as follows: four capsules of Girotate plus one capsule of Acceler and one capsule of Destagnate.
4. If the headache involves the frontal as well as temporal areas and a clinician cannot distinguish the relative dominance of the Gallbladder-Liver or Spleen-Stomach systems, the harmonizing approach may then be tried by simultaneously employing a Spleen-Stomach formula with a Gallbladder-Liver formula. If there is significant Qi deficiency, it may be advisable to choose Diamonex and Pancremax. If there is no significant deficiency in Qi, a combination of Girotate with either Octagen or Serena might be useful.
   C. Headaches of Muscular Origin

This group of headaches refers to headaches initiated by traumatic injuries of the neck or shoulder acutely or chronically via repetitive strain. Such headaches can be bilateral as well as a unilateral, and may at times cause stomach upset mimicking symptoms of migraine.

Algorithm for Managing Headaches of Muscular Origin:

1. Start with Cephalicon and continue for two weeks at regular dosages, i.e. five capsules two times a day, if no untoward effects are discernible. If the formula benefits the patient, continue with the regimen.
2. If the symptoms are specifically induced by an identifiable event such as a fall or automobile accident, the muscles and tendons are usually involved and Diamonex plus Detonin may be appropriate. Sometimes Acceler is also added to mobilize the Qi within the Gallbladder-Liver system to open up the channel. As in other clinical situations, the primary formula Cephalicon may be suspended while trying this new approach.
3. If the headache is considered to be related to repetitive strain, it is usually accompanied by other symptoms such as pain and paraesthesia in the upper extremities. Individuals with phlegm syndrome are unusually susceptible to the development of repetitive strain. So the formula to be used must also take into account the underlying imbalance in these individuals' constitution. In other words, phlegm must be transformed. For this purpose, Girotate and Pancremax are invaluable. Furthermore, the majority of such patients have a cold constitution and Kidney Yang deficiency. Noren and Cosmosol may also be useful if the patient's constitution is also too wet, and the addition of Acceler and Destagnate may also be indicated.
   D. Miscellaneous Conditions of the Head and Face

According to modern medical diagnoses, painful clinical entities that affect the head and face are rather numerous: atypical facial pain, trigeminal neuralgia, sinus headaches, dental pain of various origins, and nasopharyngeal carcinoma, to name just a few. According to TCM, however, these are all related to a disturbance of the free flow of Qi in the head and face area, and the therapeutic approaches generally revolve around the reestablishment of the local harmony. Since pain that results in a blockage of Qi flowing in the meridian is caused either by too much or too little Qi, the intervention is rather straightforward.

Algorithm for Managing Painful Conditions of the Head and Face:

1. In order to reinstate homeostasis in the head and face, it is necessary to direct the effects of the herbal agents towards those areas. This can be accomplished by two formulae: Cephalicon and Nasoclear. The use of Cephalicon is more appropriate for the head region in general whereas Nasoclear is more suitable for the frontal area as well as the oral cavity, nasal cavity, and pharyngeal area. Regardless of where the pain is located in the head, whether in the parietal, temporal, frontal, or even the occipital area, it is logical to use Cephalicon first because of its generally regulating effect on the head.
2. Next, determine whether the pain is hot, cold, deficient, or congestive and employ the appropriate modifiers if the clinical assessement gives rise to a better understanding of the underlying cause—Arctin to cool, Heleon to warm, Acceler to mobilize Qi, Destagnate to transform phlegm, and Detonin to open up blood stasis.
3. If the pain originates from an area under the control of Nasoclear, it may be better to skip Cephalicon and employ this formula instead. For instance, if facial pain in a patient arises from an infection of the maxillary sinuses, Nasoclear may turn out to be a more specific formula to use than Cephalicon. In the presence of thick purulent discharge, Destagnate may be added. It goes without saying that antibiotics may also be required if clinically indicated. In fact, the combination of modern medical and TCM approaches may be more beneficial than either approach alone.
4. Assess the patient as a whole and determine if other measures, diagnostic as well as the therapeutic, are necessary. For example, trigeminal neuralgia of sudden onset is sometimes associated with malignancy. Even when malignancy is absent, it may be indicative of an undercurrent of physiological derangement that may be mitigated by the herbal approach. For example, dental pain sometimes persists despite the best management modern dentistry can offer. This often happens in someone with a cold, Yang deficient constitution. These patients may substantially benefit from herbal formulae with a warming nature such as Noren and Lumbagon. It may seem odd at first that Lumbagon can actually be indicated for dental pain; but according to TCM, teeth are under the control of the Water element, which relates primarily to the kidney system, which in turn is also intimately related to the low back where Lumbagon exerts its regulating influence. Additionally, Lumbagon is warming in nature so it can counteract dental pain of cold nature.
   Low Back Pain

Low back pain is one of the most frequent complaints during physician office visits, yet the mechanisms of causation for low back pain remain highly elusive. According to several studies, the exact cause of low back pain cannot be ascertained in over 90% of the cases. From the viewpoint of TCM, this is rather understandable, for any interruption of the flow of Qi due either to congestion of deficiency will lead to pain. The low back is considered to be the exterior shell of the kidney, and any impairment is reflected in this shell composed of muscles, tendons, ligaments, bones, blood vessels, and nerves. All these anatomical structures are supplied by a few important energy channels or meridians, namely, the Kidney, Bladder, and Liver meridians, as well as the Governor Vessel (Du Mai).

Any injuries to the anatomical components of the low back will activate the meridians traveling through them. Conversely, disturbed energy flow in any of the meridians will bring about symptoms, perceived by the patient as rising out of these anatomical structures. For instance, an overall depletion of Kidney Qi will reduce the flow of Qi along such energy channels, resulting in stagnation or blockage. This is perceived by the patient as tiredness, weakness, or even pain. A period of mental stress without any physical injury to the low back can impede the flow of Kidney Qi in the low back, causing low back pain. Other serious medical conditions can also chronically deplete the kidney Qi and lead to low back ache. Under these circumstances, the exact cause of the low back pain cannot be easily isolated using diagnostic criteria based on modern medicine. Due to the fact that the Kidney system is the main supplier of the body's vital energy, any kind of stress and strain, whether acutely or chronically imposed on this system, will lead to low back pain.

Algorithm for Managing Low Back Pain:

1. Lumbagon should be the first line formula since it specifically directs the herbal effects to the lower back, taking into account blood stasis, phlegm, and Qi deficiency simultaneously. Lumbagon is warming overall in nature. Since most backaches are caused by Qi deficiency, particularly Yang deficiency, this formula is helpful in the majority of cases of low back pain. Naturally, this formula will be less helpful for backache associated with a significant degree of Yin deficiency. In this instance, Arctin may be added to the formula to cool it down.
2. As mentioned above, many backaches are accompanied by kidney Qi deficiency; so the employment of Neptonin and Noren would be quite reasonable, since both of these formulae tonify both Kidney Yin and Kidney Yang. These two formulae can be used together in a half-and-half mixture or, used alternately at different times of the day. Each formula may also be used exclusively depending on whether the condition is associated with predominantly Yin deficiency or Yang deficiency as verified by TCM diagnoses.
3. When the condition does not adequately respond to a trial of Lumbagon for two to three weeks, or when there is a clear-cut history of trauma (even if in the remote past), or when the painful area in the low back is located laterally, there is a good likelihood that the Gallbladder-Liver system is also involved. In contrast to popular beliefs held by the lay public as well as many medical professionals, the majority of back pain resulting from trauma originates from muscles and tendons rather than bones and disks. Since the Gallbladder-Liver system controls muscles and tendons, its contribution to the generation of chronic pain in the low back cannot be ignored. Diamonex would be a very useful formula for these conditions. If the condition is the consequence of acute trauma, or if the pain is chronic, chances are that blood stasis is present. Adding Detonin to Diamonex would be quite reasonable, particularly if signs of blood stasis are discernible.
4. If there are heat signs observed in the Gallbladder-Liver system, Diamonex may be mixed with Octagen or Serena to lower the temperature of the formula or Detonin may be added to Octagen or Serena instead of adding Detonin to Diamonex.
5. If the low back pain is caused by excessive cold, the inclusion of Heleon in a formula like Lumbagon may be appropriate. For example, ankylosing spondylitis and psoriatic arthritis are usually cold in nature. So using a warm formula, such as Lumbagon along with a heat generating modifier such as Heleon may be necessary to magnify the beneficial effects.
6. Eumensa, a formula primarily used to regulate functions in the pelvic region, may also be considered in some cases of low back pain. Pelvic conditions often give rise to low back pain, simply because both the low back and the pelvic region share the same neighborhood, namely the lower focus or the Lower Jiao of the body. Eumensa alone or in combination with Lumbagon, may be a good choice for patients suffering from chronic back pain exacerbated by menstruation and accompanied by abdominal cramps. One reasonable approach involves giving the patient a daily dose of Lumbagon throughout her entire cycle to tonify as well as regulate the Liver and Spleen system in the Lower Jiao, only to switch to Eumensa for a week to 10 days before the onset of menses (see also under Chapter 10 of Section II, Dysmenorrhea).
   Gastrointestinal Disorders

When it comes to Western modern medicine, there are numerous clinical entities associated with the GI tract. Traditional Chinese Medicine approaches diseases of the gastrointestinal system in a much more simplified fashion, based on the eight categories of diagnoses, i.e. hot, cold, congestive, deficient, Yin, Yang, exterior, and interior, regardless of the modern medical diagnoses. TCM diagnoses are based on symptoms. The element that controls GI functions is Earth. Simply put, too much or too little Earth will lead to a diverse range of symptoms. The Earth element becomes hyperfunctional when there is too much Fire or Earth, whereas too much Metal, Water, and Wood cause the Earth element to be deficient.

By narrowing down the types of abnormal elemental shifts that cause imbalance within the system, the TCM practitioner may hone therapeutic interventions quite accurately. The follow-up question to be asked by the clinician is what constitutes the pathological changes that may stem from food accumulation, excessive dampness, excessive fire, etc. This exercise will allow the physician to devise therapeutic strategies to counteract deranged physiological parameters. Since it would require extensive discussion to cover the wide spectrum of diseases of the GI tract, only a handful of common GI disorders will be used here to illustrate the logic of the herbal methods in traditional Chinese medicine.

A. Peptic Ulcers and Ulcer-like Diseases

Excellent therapeutic regimens of triple therapy have been available for some time to treat peptic ulcers with a high degree of success. However, there are still cases resistant to therapy and recurrence of disease despite successful initial treatments. More importantly, and much more frequently, many individuals with symptoms similar to those of peptic ulcers do not have readily identifiable pathology via endoscopic examination. These patients often rely on modern drug therapy for symptomatic relief, sometimes indefinitely. In these patients, TCM may offer some viable alternatives.

Algorithm for Managing Peptic Ulcers and Ulcer-like Diseases:

1. Girotate is the first-line formula of choice because of its overall simultaneously tonifying and regulating properties on the GI tract. For one reason or another, the parietal cells in the stomach or duodenum are fixed at a high set-point of acid production. Mental and emotional stress is often associated with peptic ulcers. Once again, the disease process may be hot or cold. Stress generally activates the Gallbladder-Liver system or the Wood element, which suppresses the Earth element represented by the stomach. So a hyperactive Gallbladder-Liver system will suppress the stomach, causing it to malfunction. There may be too much Water and Wood or too much Wood and Fire. Although both such states are replete with Wood, the former is cold while the latter is hot. In these situations Girotate maybe modified with Heleon or Arctin to normalize the underlying imbalances.
2. If the condition is caused by excessive Water (acid secretion) in the upper GI tract and the body's constitution is cold and wet as described by the drink syndrome, characterized by a pale and wet looking tongue and other signs that are consistent with this diagnosis, then Pancremax may be a very useful formula.
3. Destagnate may also be an important modifier to consider if phlegm figures prominently in the pathology. Thick futrry coating in the middle of the tongue often signifies such condition.
4. Although Girotate contains Qi mobilizers in its formula, it does not emphasize on Qi mobilization, as Qi mobilizers are not the major components of the formula. When epigastric pain is a prominent feature of the condition, the addition of Acceler to Girotate may be of value.
5. When a stressful lifestyle seems to play a very important role in the pathogenesis of the ulcer-like symptoms or ulcers, Relaxol may be indicated, since this formula specializes in harmonizing the liver and stomach. Relaxol is suitable for conditions that are neither hot nor cold. Serena, on the other hand, concentrates on cooling and calming the Gallbladder-Liver when the ulcer or ulcer-like condition is associated with heat signs. It is, of course, also reasonable to use either Heleon or Arctin to adjust the temperature of the formula.
6. Many of the GI conditions are actually caused by an overactive Gallbladder-Liver system. Therefore, it is also possible to manipulate and adjust the activity of the Gallbladder-Liver system to lighten the burden on the Spleen-Stomach system. Octagen directs its effects to the Gallbladder-Liver, so adding a bit of Detonin to this formula can sometimes relieve the trapped energy represented by the Wood element.
   B. GERD

Gastroesophageal reflux disease or GERD is extremely common as well as troublesome to treat. The therapeutic interventions in modern medicine are mainly palliative and often ineffective, so patients are sometimes required to change their lifestyle, such as by propping themselves up at night when they go to sleep.

The symptoms of reflux may be viewed as a reversal of Qi according to traditional Chinese medicine, as normal Qi should flow downward as the peristalsis of the esophagus propels food towards the stomach. When the normal flow of Qi is blocked, it is turned back on this path, causing retrograde flow of fluid from the stomach, hence the reflux. Over-eating sometimes leads to reflux, as the stomach is so full that it cannot accept any more food, which is denied admission to the stomach and sent back up the esophagus. Congestion of stomach Qi will duplicate this phenomenon. So the patient with a constitution that is too rich in Earth element (resulting from to much Earth or too much Fire) is prone to develop reflux diseases.

On the other end of the spectrum, if the Stomach Qi is too weak, its capacity to accept food is substantially reduced. It is as if the stomach becomes smaller, thus getting full sooner and more easily and tends to reject food for entry, again causing reflux. Weak Stomach Qi, or too little Earth element is often found in a constitution that is rich in Metal and Water or Water and Wood. In other words, a stomach that is congested is like a normal sized stomach that has been preloaded with food, while the stomach that is deficient is like the stomach that has been reduced in size. In either case, reflux may be a problem.

Algorithm for Managing GERD:

1. Due to the regulating effects of Girotate on stomach functions, it should be a first-line formula. If the formula does not produce any immediate beneficial effect, there should be no cause for concern if it does not produce any undesirable effects. Stay with it for a few weeks before switching to another formula, unless the clinical findings based on the analysis of the patient's symptoms and signs derived from pulse diagnosis and tongue diagnosis point to a better formula.
2. If the patient's constitution is cold and wet with the tongue appearing wet and pale, with perhaps reduced coating in the middle of the tongue, and the right middle pulse is weak, indicating stomach Qi deficiency, then Pancremax may be indicated.
3. If the symptoms of reflux are a result of Stomach Qi congestion, as may be seen in a patient with thick white or yellow coating in the middle of the tongue, then the use of Pancremax may be counterproductive. In this case, the stagnant phlegm or congestion in the Stomach system needs to be dispelled by the use of Destagnate, or Destagnate and Acceler. Although Girotate still can be used, it would then basically function as a guidance system, while the emphasis would be on dispersion. Since Girotate possesses tonifying functions as well, the ratio between tonification and dispersion must be changed in favor of dispersion. This may be accomplished by prescribing, for instance, three capsules of Girotate along with two capsules of Destagnate two times a day. Such a ratio can be adjusted according to the degree of stagnation.
4. Stress along with strong emotions can often be linked to GI distress as with any “gut wrenching experience”. Stress activates the Gallbladder-Liver system (the Wood element), which then suppresses the Spleen-Stomach system (the Earth element). Besides focusing on the Stomach when dealing with upper GI symptoms, one must not ignore the role played by the Gallbladder-Liver system. Several Liver-oriented formulae such as Octagen, Diamonex, Serena, and even Relaxol can come in handy in the management of GERD. When the Stomach energy is excessive, tonification of the Liver system is a countermeasure in a “back door” sort of way. In this case Diamonex is a reasonable candidate. If, on the other hand, the Stomach Qi is weak, suppressing the activity of the Gallbladder-Liver system by harmonizing it with Octagen or calming it with Serena may get some pressure off the Stomach system.
5. GERD may be associated with either hot or cold syndromes: hot if too much Fire and Earth, and cold if too much Wood and Water. For example, adding Heleon to make the formula warmer or Arctin to make it cooler may also be helpful.
6. If there is a constant war between the Stomach and the Liver, and one cannot be sure which system is winning, causing the symptoms of reflux, a good way to hedge is by giving the patient Relaxol and observing the clinical response.
What is the common denominator for GERD, nausea, and vomiting? The answer is reversal of Qi. Obviously, vomiting involves reverse peristalsis or reversal of Qi. Reflux may be considered as mini-vomiting. Nausea may be thought of as the threatened reversal of Qi. Even though they are considered different symptoms, they basically share the same basic pathogenic mechanism. The same strategy used to manage GERD is equally applicable when dealing with nausea and vomiting.
   C. Diarrhea and Constipation

Diarrhea may be hot or cold, constipation may be hot or cold, and irritable bowel syndrome may also be hot or cold or alternating hot and cold. Diarrhea can often be hyper-secretory, interpreted by Chinese medicine as being wet or damp, which in turn may be associated with heat or cold. Constipation can also be due to excessive heat or excessive cold, Qi congestion, or Qi deficiency. Likewise irritable bowel syndrome can be either hot or cold. Certain types of irritable bowel syndromes can be related to a hyperactive Gallbladder-Liver system, especially when the symptoms vacillate between diarrhea and constipation.

Algorithm for Managing Diarrhea:

1. Girotate should be the initial main formula. It may be indicated in some types of acute diarrhea such as travelers' diarrhea. Its ability to tonify and regulate the entire GI tract makes it especially useful in chronic diarrhea.
2. If the diarrhea consists of watery stools, and its odor is not too strong, as when the patient is suffering from a cold condition, then Pancremax is a better choice. Cold diarrhea is often found in patients suffering from ulcerative colitis, for instance. Girotate with Heleon or Pancremax with Heleon may also be tried.
3. If the diarrhea consists of foul-smelling stools, and is associated with a burning sensation in the anal area, accompanied by thick yellow coating on the tongue especially near the base, the condition is usually caused by damp heat. Arctin and Destagnate are to be added to Girotate for such a condition.
4. Individuals who have chronic diarrhea are generally Kidney Qi deficient. Noren may be used to tonify the Kidney Qi. Cosmosol may be also useful for global tonification.
5. An overburdened GI tract such as the one found in food accumulation syndrome can also cause diarrhea. If the subjective symptoms and objective physical signs point to such a diagnosis, the addition of Acceler and Destagnate to a formula such as Girotate may be in order.
6. An overly active Gallbladder-Liver system can also weaken the GI functions leading to the symptoms of diarrhea. In this case, the Wood element needs to be reduced by employing a formula such as Octagen, or Octagen with Detonin.
   Algorithm for Managing Constipation:
1. Constipation may be divided into two major camps: a hot type and a cold type. The large intestine belongs to the element of Metal. When the energy of Metal is excessive, the functions of large intestine are impaired, paralyzed with too much energy, and constipation results. On the other end of the spectrum, if there is too much Fire suppressing the element of Metal, the deficiency of Metal may also lead to constipation. Since Girotate has the ability to normalize the entire GI tract, it is logical to try it first. Either Heleon or Arctin may be added to the formula, depending on whether the constipation is cold or hot. Elderly patients tend to have Qi deficiency, and tend to suffer from cold syndrome, so the warming effect of the combination of Girotate and Heleon may be desirable. In some instances, however, when the elderly may also suffer from constipation of the hot type, one must therefore determine the nature of the condition by using the tools of TCM such as pulse diagnosis and tongue diagnosis.
2. Constipation or absence of bowel movements sometimes is indicative of serious illness such as intestinal obstruction, according to modern medicine. TCM not only recognizes this, but includes other conditions, some serious enough to be life-threatening, collectively known as stomach congestion syndrome. This is associated with a board-like abdomen, inability to defecate, high fever with delirium, and excessive sweating, all due to marked heat entrapment in the Yang Ming or Stomach system. In such instances, much stronger and definitive formula such as the Grand Riding Qi Tang must be used. Girotate may in fact be contraindicated in this situation.
3. If the constipation is caused by damp heat, a heavier dose of Arctin with a moderate dose of Destagnate under the guidance of a small dose of Girotate may be useful. Try two capsules of Girotate, with two capsules of Arctin, and one capsule of Destagnate.
   D. Irritable Bowel Syndrome

Alternating constipation and diarrhea is a hallmark of IBS, although the diarrhea component is usually more troublesome. Tension, such as that before making a public presentation, may promptly trigger an episode of diarrhea. Since stress originates from the Gallbladder-Liver system, the activation of the Wood element suppresses the Earth element, which influences the lower GI tract, thus giving rise to diarrhea. According to a study in North Carolina, an extremely high percentage of patients with difficult to manage IBS give a history of sexual abuse. Since the dominant emotion of the sexually abused is anger, an emotion closely affiliated with the Gallbladder-Liver system, such clinical findings should pose no surprise at all to a student of TCM.

Algorithm for Managing Irritable Bowel Syndrome:

1. The target for intervention should probably be the Gallbladder-Liver system, which must be modulated with the various formulae acting on the system. Such formulae include Octagen, Diamonex, Serena, and Relaxol, perhaps along with adjustments by the various modifiers, as may be indicated after fine-tuning the diagnosis.
2. The Earth element, namely the stomach, may also be used to counterbalance the influence from the Gallbladder-Liver system. As mentioned earlier, the stomach and liver are in perennial antagonism; the ultimate balance between the two can be achieved by increments or decrements on either side. So the other approach to treat IBS is to take the Earth element route, which encompasses such formulae as Girotate, Pancremax, Nasoclear (activating on Earth and Metal), along with appropriate modifiers to cool, warm, mobilize, or decongest.
   Allergies:

According to modern medicine, allergic reactions are due to the sensitization of the body to allergens or antigens, therefore therapeutic interventions may involve the administration of incremental doses of the allergens to allow the body to get used to them, so as not to react to them. Many patients suffering from allergies in fact have multiple allergies. What modern medicine fails to recognize so far is that the immune system may be to blame. The immune system tends to be overly excitable and irritable. This kind of behavior of the immune system may be more accurately characterized as the “irritable immune syndrome”.

It has been long recognized that patients with atopy can become allergic to many substances and these same patients, when scrutinized with TCM diagnostics, often suffer from General Qi deficiency, and are often shown to have Kidney Yang deficiency. When people are sick and tired, they become irritable. Women can become quite irritable during menses and menopause when they suffer from a more abrupt depletion of Qi. So irritability is in fact a defense mechanism set up to protect the person who is physically weak, thus serving as a warning to would-be attackers.

In multiple allergies, the allergy-stat may be set too high, as a result of Kidney Qi deficiency. Consequently, when Kidney Qi deficiency is mitigated by either herbal remedies or acupuncture, sensitivities to different allergens tend to abate across-the-board, sometimes disappearing completely.

Two Kinds of Allergy Will be Discussed:

1. Upper respiratory allergies such as allergic rhinitis and bronchitis
2. General allergies such as skin allergy, food allergy, and multiple chemical sensitivities.
   A. Upper Respiratory Allergies

Seasonal allergic rhinitis or hay fever, perennial rhinitis, chronic sinusitis, and allergic bronchitis all involve the upper respiratory tract, which is controlled by the Metal element according to TCM. Associated with these conditions is the global factor, which has been discussed above in relation to Kidney Qi deficiency, and the local factor stemming from a disturbed homeostasis in the upper respiratory tract. According to TCM, the lung is a very fastidious organ, tolerates little deviation from the norm, and is particularly sensitive to environmental changes. So a little change means a lot of change to the lung, regardless of whether the nature of change is hot, cold, congestive, or proficient.

Algorithm for Managing Seasonal Allergies:

1. The formula Nasoclear is specifically designed to reestablish the internal balances in the upper respiratory areas. It has the components to tonify the Stomach (Earth element), which in turn provides the impetus to mobilize the Metal element as well as to regulate it. This should be a first-line formula. However, its effect sometimes may not be fully appreciated until after a couple months of continuous administration.
2. The global tonification formulae such as Noren, Neptonin, or Cosmosol may be used to provide general tonification. Nasoclear and one of these formulae may be administered in an alternating schedule: for example, five capsules of Nasoclear two times a day for one month, followed by five capsules of Noren two times a day for another month. Alternatively, the two formulae may be combined and administered together at reduced dosages such as three capsules of Nasoclear with three capsules of Noren two times a day.
3. Certain hay fevers, especially the ones occurring in spring and early summer, may be considered by TCM as being syndromes of wind and heat, particularly when accompanied by itchy and tearing eyes. When the diagnosis of wind heat is verified by pulse diagnosis and tongue diagnosis, it may be necessary to add Arctin to the formula. And if the patient has a Yin deficient constitution, he may benefit from taking Neptonin as well. Again, when Nasoclear is taken together with Neptonin, it is best to reduce the dosage for each accordingly. Neptonin can also be given way before the arrival of the allergy season to regulate the Yin deficient constitution of the patient.
4. The majority of perennial rhinitis cases are often associated with cold conditions caused by a Yang deficiency state. In these cases, Heleon may be added to Nasoclear. Noren, instead of Neptonin, may also be indicated as a tool for global management, which may at times require several months to complete, especially when positive response is apparent.
   B. General Allergies Including Multiple Chemical Sensistivities

General allergies, as discussed earlier, are often related to Kidney Qi deficiency states: Yin deficiency, Yang deficiency, or deficiency in both Yin and Yang.

Global allergy, as seen in cases of multiple chemical sensitivities, often gives rise to multiple somatic complaints such as fatigue, insomnia, depression, generalized muscle pain, and so on. It is simply impractical to try to desensitize patients from all the allergies. Instead, the patient's allergy-stat should be lowered by the various means available in TCM. To extrapolate the principle of equivalence between Kidney Qi deficiency and the hyperallergic states or immunological irritability, it is not difficult to imagine that a number of the so-called autoimmune diseases are also found in patients with an “irritable immune system”, which is closely related to significant Kidney Qi deficiency. As a matter of fact, it has been shown in China that many chronic conditions characterized by Kidney deficiency such as atherosclerosis, bronchial asthma, scleroderma, anovulatory uterine bleeding, and systemic lupus erythematosus significantly improve with either Kidney Yin or Kidney Yang tonifier or both.

Algorithm for Managing General Allergies:

1. These various deficiency states may be dealt with by employing Noren, Neptonin, Cosmosol, or combinations in various proportions of these formulae, subject to clinical assessment based on TCM.
   C. Skin Allergies

Dermatological allergies such as certain eczematous conditions may either be associated with excess Metal or insufficient Metal, since Metal relates to Lung, which is the system in charge of the skin.

Algorithm for Managing Skin Allergies:

1. When there is Metal excess in the body's constitution, the patient generally suffers from cold conditions which may be alleviated by warm formulae. For instance, adding Heleon to Diamonex will engender the elements of Wood and Fire, thus suppressing the Metal element.
2. On the other hand, a patient who has an innate deficiency of Metal tends to have a hot constitution, which may be toned down by formulae of cooling nature. Nasoclear does tonify Earth and Metal, so it may be used in this situation. The addition of a cooling modifier such as Arctin may make Nasoclear even more effective. It should not be surprising that a formula such as Nasoclear may be helpful in skin conditions, because both the nasal passage and skin are controlled by the Lung system. Furthermore, it is a well established clinical fact in modern medicine that there is a close linkage between asthma and eczema in an atopic individual.
   D. Food Allergies

Food allergies are in many ways similar to respiratory allergies, except individuals with respiratory allergies have problems with Metal, whereas individuals with food allergies have problems with Earth, which is either deficient or excessive. Therefore, to modulate the response of the gastrointestinal tract to the various substances that cause allergies, the Earth element needs to be regulated.

Algorithm for Managing Food Allergies:

1. Girotate may be tried first, due to its overall regulating effects on the GI system. Patients with multiple food allergies, as in those suffering from chronic fatigue syndrome, often experience malfunctions of their gastrointestinal system. They are often found to have yeast infections in their intestinal tract because of impaired immune response in their digestive systems. Simple tonification and regulation of their Spleen-Stomach systems with Girotate may be of great benefit.
2. If the patient also suffers from a cold constitution with excessive amount of cold phlegm (dilute phlegm), as evidenced by a great deal of moisture and pale appearance of the tongue, then Pancremax may be more appropriate. Alternatively, Girotate plus Heleon may also work.
3. If General Kidney Qi deficiency is found, the triad of tonification formulae including Noren, Neptonin, and Cosmosol (or combinations thereof) should be considered.
4. If the patient with food allergies also suffers from upper respiratory allergy, Nasoclear, which acts on both the Metal and Earth elements, or Lung and Stomach, may also be a logical choice to kill two birds with one stone, so to speak.
5. Depending on the diagnostic analyses, other members of the M Series may be enlisted to assist the main formulae mentioned above to achieve the desired goals.
   Dysmenorrhea

Abdominal pain or cramps associated with menstruation has plagued women of childbearing years for ages. This symptom accounts for one of the most frequent primary complaints presented at visits to physicians The symptom can be so disabling that it often deals a severe blow to the quality of life and productivity. Modem pharmacological therapy consists of pain medications and hormonal therapy. Though such approaches may alleviate the symptoms, they may not be able to remove the body's underlying proclivity to develop such symptoms due to the internal imbalances in these women.

From the viewpoint of modern medicine, the monthly cycle of menstruation is, of course, driven by the fluctuating levels of reproductive hormones. Traditional Chinese Medicine, on the other hand, considers menses to be a phenomenon resulting from the ebbs and flows of Qi and blood governed by the Kidney and Liver systems. Generally speaking, Qi surges upward at the time of ovulation and becomes relatively depleted at the time of menstruation. Consequently, menstruating women experience a cyclical dwindling of Qi on a monthly basis and it is the Qi depletion that constitutes the root cause of the multitude of symptoms surrounding menstruation.

According to the TCM tenets, “when Qi is blocked, pain ensues”. When the amount of Qi in the channels is diminished, its forward motion is impeded, causing it to stagnate, much like a dried up canal no longer being effective in transporting boats along its course. This impedance or blockage produces pain. The elderly population suffers from more pains and aches because, as a whole, they suffer from general Qi deficiency. However, a blockage may not always be associated with Qi deficiency, as stagnation may be the result of blood stasis or excessive phlegm, including food accumulation. In this instance, the blockage is similar to a boulder in the middle of the river, preventing it from flowing freely, even though the amount of flow in the river is normal. But if the flow is low, the problem gets compounded. For example, a woman with a blood stasis syndrome such as endometriosis tends to have more severe menstrual cramps due to the additive effects of the blockage in the presence of Qi deficiency.

Algorithm for Managing Dysmenorrhea:

1. The formula Eumensa is composed of a healthy dose of Qi tonifiers, particularly for the Liver and Kidney, along with Qi mobilizers for the lower focus of the body. Therefore, it is the ideal first choice to reestablish homeostasis in that region of the body. This formula may be administered in several ways:
   a. Episodically: the formula may be started approximately one week prior to the onset of menstrual flow and continued through the first couple of days of menses or until menstrual cramps cease for that particular period. This regimen is then repeated for several menstrual cycles in the same fashion.
   b. Continuously: when dysmenorrhea has been a long-standing problem, recurring every cycle and severe in nature, Eumensa may be administered throughout the entire cycle in an effort to bring about a stronger harmonizing effect to the pelvic region.
2. Since dysmenorrhea occurs often in the backdrop of Kidney Qi deficiency, the enlistment of Neptonin, Noren, and even Cosmosol should be able to enhance the Qi. The specific choice of formula may be determined by the total clinical picture. For example, if there are quite a few heat signs due to Yin deficiency, Neptonin is appropriate. If Kidney Yang deficiency dominates, Noren may be indicated. If there is an abundance of heat signs and symptoms, Arctin may be added to Neptonin. Likewise, if Yang deficiency is more severe and there are a lot of cold symptoms, then Noren plus Heleon or Neptonin plus Heleon may also be considered. Sometimes a small amount of Acceler can also potentiate the effects of the above formulae.
3. If the patients' constitutions are dominated by the Water element as in the case of many patients suffering from polycystic ovary syndrome (PCOS), a formula that is strong on the Earth element such as Pancremax may be used to suppress the Water excess.
4. Many patients with menstrual cramps also suffer from low back pain, because the low back is considered by TCM to be the housing or envelope of the Kidney system, the deficiency of which leads to both abdominal pain and low back pain. Lumbagon, useful in the management of low back pain, will also have distinct effects on the lower abdomen, which is also part of the lower focus or Lower Jiao.
5. As mentioned in the previous sections, the pelvic region is also governed by the Gallbladder-Liver system, the deficiency or excess of which is equally likely to be manifested as menstrual cramps. For Liver deficiency, use Diamonex; for Liver stasis, use Detonin with a guidance formula to the Liver system such as Octagen, Diamonex, or Serena. Once again, the addition of Acceler may complement the actions of these formulae.
6. The Merideon formulae may be combined in multiple ways to cope with the underlying physiological derangements to promote the health of the patients with this common disorder. To a large degree, how the formulae are combined to suit a particular patient should be determined by the diagnostic acumen of the physician. History, pulse diagnosis, tongue diagnosis, laboratory findings and other clinically relevant facts should all be taken into account.
Caution: Eumensa contains a small amount of anti-blood stasis agents, counterbalanced by a heavy dose of tonification agents. As a result, the Qi consumption effect is minimal. Still, this formula is not recommended for use during pregnancy.
   Painful Conditions of Muscular Origin:

Traditional Chinese Medicine considers any blockage of Qi to be a possible cause for pain, regardless of the nature of the blockage. This blockage may be hot, cold, deficient, congestive, or damp; it may also originate from the interior or exterior and be caused by trauma, both physical and emotional, inclement weather, poor dietary habits etc. The onset of pain may be gradual or abrupt. The classification of painful conditions according to TCM is quite different from that of modern medicine. Nevertheless, for the purposes of discussion, the Western diagnoses will be used to illustrate the thinking process of TCM.

A. Fibromyalgia

Fibromyalgia is a unique type of muscular pain characterized by generalized body ache and the presence of multiple trigger points; it is often associated with other comorbidities such as insomnia, depression, fatigue, and is notoriously difficult to treat with modern medicine. Recent research has found increased levels of substance P in the cerebrospinal fluid of patients suffering from fibromyalgia as compared to normal subjects.

From the viewpoint of TCM, fibromyalgia is a cold and wet syndrome associated with Kidney Qi deficiency. The Water element dominates in the constitution of these patients. The more excessive the Water element, the more likely the patients are suffering from the drink syndrome, the type of phlegm syndrome where the phlegm is more dilute than tenacious. Drink syndrome may precipitate symptoms such as dizziness, insomnia, inability to concentrate, palpitations, chronic cough, anxiety, or even panic attacks, in addition to the symptoms of pain and fatigue typical of dampness or phlegm syndrome.

Algorithm for Managing Fibromyalgia:

1. Since a majority of patients with fibromyalgia suffer from Kidney Yang deficiency, as evidenced by a pale looking wet tongue, often with teeth marks (scalloping) on the lateral aspects of the tongue, and are frequently associated with a deep slow pulse, the formulae of choice is Noren. Cosmosol may also be used because of its general Qi tonification properties.
2. To control the excessive Water associated with this syndrome, a formula that is rich in Fire and Earth element, such as Pancremax, may come in handy. The underlying physiological derangements of patients suffering from chronic fatigue syndrome of the Yang deficiency type oftentimes overlap those of fibromyalgia to a great extent; thus, the TCM management approach is often the same for both conditions. Pancremax is especially useful where there are significant symptoms of Stomach and Spleen Qi deficiency. Pancremax may be viewed as the nemesis of the dilute phlegm or drink syndrome due to its ability to counteract many of the physiological effects of excess Water.
3. Since the lack of Fire element in fibromyalgia patients is obvious, Diamonex alone may be utilized to generate Wood, which in turn generates Fire; or the fiery modifier Heleon may be combined with Diamonex to produce heat and Fire together. A better functioning Gallbladder-Liver system can also be more effective to channel the Water into the Wood element.
4. Since most patients with fibromyalgia have a significant decrease of Kidney Qi, which sometimes manifests itself in the exterior shell of the Kidney as low back pain, the formula Lumbagon may be extremely useful in regulating the internal environment of the Kidney system. Besides, there is an herbal component in the formula that dispels phlegm, a prominent feature of fibromyalgia, and another component that acts on the Gallbladder-Liver system to clear blood stasis, a problem that is also commonly encountered in fibromyalgia.
   B. Repetitive Strain and Carpal Tunnel Syndrome

These two syndromes are discussed together because the underlying pathology from the viewpoint of TCM is more or less the same for both conditions. From the standpoint of modern medicine, it is the cumulative effects of microtraumas that lead ultimately to the syndromes. Nevertheless, individuals subject to the same workload or repetitive strain may not necessarily develop symptoms. Apparently, some people are more predisposed to develop such syndromes. It has been observed in recent clinical studies that obesity and the female gender are risk factors. Women as a whole are more Yin than men and there is a close linkage between obesity and phlegm. Patients with repetitive strain syndrome and fibromyalgia share many common underlying characteristics such as excessive phlegm or the dominance of the Water element in their constitutions. Although these conditions can be treated with acupuncture with positive results, the herbal approach can make patients more responsive to acupuncture.

Algorithm for Managing Repetitive Strain Syndrome and Carpal Tunnel Syndrome:

1. According to the principles of Chinese medicine, phlegm is primarily generated in the Stomach. Weakened Stomach Qi allows phlegm to stagnate, causing a variety of problems. Another well-established principle of TCM clearly states that the Spleen system controls all four extremities. This principle is particularly relevant because the upper extremities are most likely to be involved in these syndromes. Logically then, the Stomach and Spleen Qi in these patients should be strengthened. Either Girotate or Pancremax would be suitable.
2. If the phlegm syndrome is more advanced, as verified by other physical signs and symptoms such as a thick and very greasy coating on the tongue or a slippery pulse, the addition of Destagnate to the above formulae may be appropriate. More often than not, these patients suffer from simultaneous Qi deficiency and stagnation. Tonification alone sometimes increases the stagnation, while dispersal of phlegm alone may deplete Qi. A balance must be struck between Qi tonification and phlegm dispersion to achieve the optimal beneficial effects. This may be achieved by adjusting the ratio between the tonification components and dispersion components in a formula.
3. Acceler, the Qi mobilizer of the M Series, may also have a role to play, for it too can potentiate the action of Destagnate for the purpose of phlegm dispersion. It should be kept in mind, however, that Acceler is partially Qi consuming and its nature is warm. Adding Acceler to Pancremax would make the combined formula warmer than the combination of Acceler and Girotate. Adding Acceler to Girotate would make the new formula more dispersing than the combination of Acceler and Pancremax. This is due to the inherent difference between Girotate and Pancremax, although both formulae hone their actions on the Spleen-Stomach system.
4. There are occasions when tonification of the Spleen system is not quite enough because the Fire of the Gate of Life (Mingmen) is so weak that it fails to adequately supply the vital energy to the Spleen. Noren is an ideal candidate for this job.
5. Clinically, most cases of carpal tunnel syndrome have the hallmark of Yang deficiency. Repetitive strain syndrome, on the other hand, can be at times a Yin deficient condition. If significant Yin deficiency is affiliated with pain in the upper extremities, a formula such as a Neptonin may be appropriate.
   C. Post-traumatic Pain

Although bone fractures may sometimes be an important component of physical trauma, for every one patient who seeks medical attention for bony injury, there are many more post-traumatic pain patients with soft tissue injuries sustained from falls, sports, and automobile accidents. Postoperative pain and painful surgical scars also belong to this category. These painful conditions may be acute during the period immediately following injuries, subacute, or even chronic, sometimes lasting months following the initial trauma.

### Acute Pain

This kind of pain is usually managed with analgesics successfully. However, there is the current belief (or not so current belief if one subscribes to the Thalamic Neuron Theory, 1976) that acute pain may sensitize neurons in the central nervous system to set their pain-stats high in the process known as “winding up”, which may lead to chronic pain. The habituation of the central neurons to a pathological state of over-excitability is equivalent to the formation of blood stasis according to TCM.

Algorithm for Managing Acute Pain Due to Soft Tissue Injuries:

1. Soft tissue injuries can involve muscles, tendons, ligaments, nerves, and blood vessels. Acute injuries also trigger the stress response governed by the Gallbladder-Liver system. Therefore, strengthening and regulating the Gallbladder-Liver system during this crucial acute phase of injury may serve the patient well. Combining Diamonex with Detonin should probably be the initial preferred approach.
2. To promote healing, a patient's internal energy imbalance at the time of injury or shortly thereafter should be eliminated or reduced as much as possible by the employment of the modular system. Many patients become accident prone because of such energy imbalance representing a compromised state of health. For example, Noren can be used to mitigate Kidney Yang deficiency, Neptonin may be useful to counteract Yin deficiency, Cosmosol can alleviate general Qi deficiency, Relaxol can harmonize the relationship between the Spleen and the Liver, and so on.

### Chronic Pain

When pain persists long after soft tissue injuries, it assumes the characteristics of neuropathic pain, which arises not out of noxious stimuli from tissue injuries, but rather the malfunctioning sensory neurons themselves. This is modern medicine's explanation. TCM on the other hand views this phenomenon as a compromised state of health due to either deficiency or stagnation of Qi and blood.

Algorithm for Managing Chronic Pain Due to Soft Tissue Injuries:

1. The history of acute injury strongly suggests the presence of blood stasis, which relates primarily to a Gallbladder-Liver system that is either congestive or deficient. Diamonex may be used in either condition, although Detonin ought to play a role in addition to Diamonex when blood stasis is present.
2. According to TCM, when Qi stops flowing, blood stops flowing. It may be necessary to add Acceler to the formula to further mobilize the Qi. If the entrapment of blood produces excessive heat, which it often does, then further modification of the formula with Arctin may be in order.
3. As mentioned in earlier sections, a person with less than optimal health would be more likely to experience chronic pain after an injury. Normalizing this person's health can certainly promote healing. The types of formulae to be used are largely dependent on the result of diagnostic analysis based on TCM. For example, someone whose constitution is cold and wet may benefit from taking Pancremax; a patient with Kidney Yang deficiency can be helped by Noren; Yin deficiency by Neptonin, and so on.
4. Other formulae that act on the Gallbladder-Liver system such as Octagen, Serena, or Relaxol may also be included. Add modifications if necessary.
5. If several formulae may be indicated for the patient's condition, they may be combined to achieve the optimal effect, or they may be used in a sequential manner. Try to select the formula with the most coverage for all the main symptoms.
   D. Other Forms of Muscle Pain

There are numerous clinical entities that produce muscular pain. There is no single formula that can encompass these conditions. The management has to be on a case-by-case basis. For instance, certain inflammatory disorders of the muscular system such as polymyositis or dermatomyositis are often hot disorders based on TCM analysis. The formula of choice during the chronic phase may well be Neptonin or Neptonin plus Arctin. Polymyalgia rheumatica that often affects the elderly population is mainly related to Qi deficiency, or cold and wet syndrome, and its management should be similar to that of fibromyalgia.

Alternative Embodiments:

While 19 formulae are listed as members of the proposed modular system, the employment of this modular concept is not restricted by the number of constituent formulas, nor to the current ingredient lists of each formula.

Conclusions, Ramifications, and Scope

Accordingly, the reader will see that the modular system for novel Chinese herb formulas presented above allows for a limited number of herbal formulations to generate an almost unlimited array of treatments for a wide variety of medical conditions in a custom manner for individual patients.

Specifically, the modular system

(a) provides a modular system of herbal formulations which may be uniquely combined to treat a variety of medical conditions in different individuals
(b) provides a modular system with a limited number of herbal formulations to reduce storage and inventory requirements for a comprehensive herbal pharmacy
(c) provides a modular system of herbal formulations that will allow healthcare practitioners with a basic understanding of TCM to deliver effective treatments to patients without requiring intimate knowledge of individual constituent components of the formulas.
(d) provides a modular system of herbal formulations that is safe to use

Although the modular system described contains many specificities, these should not be construed as limitations on the scope of the invention, but rather as an exemplification of one preferred embodiment thereof. Many other variations are possible. For example, the employment of this modular concept is not restricted by the number of constituent formulas, nor to the current ingredient lists of each formula. Also, although one preferred embodiment involves the oral delivery of such modular formulations in capsule form (with dried herbal component agents), the formulas may be delivered in tablet or liquid form as well. In general, the essential novel strategy employed by this invention is the use of a class of general tonification formulas along with classes of specific modifying formulas to fine tune the intended therapeutic effect. By using a multi-tiered system, many more permutations of safe and therapeutic formula may be generated; more so, than if all components of a modular system were general tonifiers.

Accordingly, the scope of the invention should be determined not by the embodiments illustrated, but by the appended claims and their legal equivalents.

I claim:

1. A system for the treatment of medical conditions in humans comprising a plurality of formulas, each consisting of individual herbal, mineral, or animal components, whereby a practitioner skilled in the basic principles of Traditional Chinese Medicine may apply such formulas in a modular manner to create custom formulations for individual patients for therapeutic purposes.

2. The system of claim 1 wherein said formulas consist of one or more classes of general tonification formulas and one or more classes of modifying formulas.

3. The system of claim 1 wherein said formulas consist of one class of general tonification formulas, one class of more specifically designed general formulas, and one class of modifying formulas.

4. The system of claim 1 wherein the individual components of formulas are comprised of herbal, mineral, or animal derived agents typically used in the practice of Traditional Chinese Medicine.

5. The system of claim 1 wherein the individual components of formulas are dried herbal, mineral, or animal derived agents in capsule form.

6. The system of claim 1 wherein the formulas consist of 7 general formulas, 7 more specific formulas, and 5 modifying formulas
